(19) United States
(12) Patent Application Publication
(10) Pub. No.: US 2025/0306037 A1
(43) Pub. Date: Oct. 2, 2025

(54) METHODS TO DETECT AB PROTEOFORMS AND USE THEREOF

(71) Applicant: Washington University, St. Louis, MO (US)

(72) Inventors: Kwasi MAWUENYEGA, St. Louis, MO (US); Randall BATEMAN, St. Louis, MO (US)

(21) Appl. No.: 18/864,824

(22) PCT Filed: May 11, 2023
(86) PCT No.: PCT/US2023/021891

§ 371 Date: Nov. 11, 2024

Related U.S. Application Data
(60) Provisional application No. 63/340,689, filed on May 11, 2022.

(51) Int. Cl. G01N 33/68; A61K 45/06; G01N 30/02; G01N 30/72; G01N 30/88; G01N 33/543

(52) U.S. Cl. CPC G01N 33/6896; A61K 45/06; G01N 30/72; G01N 30/88; G01N 33/543; G01N 2030/027; G01N 2030/8831; G01N 2333/4709; G01N 2800/2821; G01N 2800/50; G01N 2800/60

(57) ABSTRACT

The present disclosure relates to methods useful to identify subjects having an increased risk for conversion to mild cognitive impairment (MCI) due to Alzheimer's disease (AD) and/or stage a subject prior to the onset of mild cognitive impairment (MCI) due to Alzheimer's disease (AD) and/or identify subjects with Aβ amyloidosis and/or to identify subjects who should or should not undergo further testing or treatment for Aβ amyloidosis, as well as methods for treating subjects diagnosed with Aβ amyloidosis by the methods disclosed herein.

### GOVERNMENTAL RIGHTS

This invention was made with government support under AG032438 awarded by the National Institutes of Health. The government has certain rights in the invention.

### FIELD OF THE TECHNOLOGY

The present disclosure relates to methods useful to identify subjects having an increased risk for conversion to mild cognitive impairment (MCI) due to Alzheimer's disease (AD) and/or stage a subject prior to the onset of mild cognitive impairment (MCI) due to Alzheimer's disease (AD) and/or identify subjects with Aβ amyloidosis and/or to identify subjects who should or should not undergo further testing or treatment for Aβ amyloidosis, as well as methods for treating subjects diagnosed with Aβ amyloidosis by the methods disclosed herein.

### BACKGROUND

Aggregation and accumulation of amyloid-beta (Aβ) in the central nervous system, particularly Aβ42, is implicated in the pathogenesis of several neurodegenerative diseases. Unfortunately, current methods for clinically defined evidence of Aβ deposition have a number of limitations. Neuroimaging studies have emerged as tools for detection of cerebral Aβ amyloidosis; however, their use is limited by expense and availability. Furthermore, dysregulated Aβ kinetics may precede imaging-based amyloid detection by many years. Decreased cerebrospinal fluid (CSF) Aβ42 levels and increased CSF tau are associated with amyloidosis and risk of progression to dementia.

Advances in high resolution mass spectrometry techniques have created new methodologies to measure the abundance of proteins in biological samples. In spite of advances in instrumentation and data analysis software, sample preparation is still an immense challenge. The choice of sample preparation method affects the observed metabolite profile and data quality, and can ultimately affect reported results. This is particularly true for proteins and peptides in low abundance in biological samples. Peptides that fall under this umbrella include many proteolytic fragments of full length proteins, which are differentially produced in various disease processes.

Accordingly, there remains a need in the art for improved sample processing methods in order to quantify low abundance, Aβ proteoforms in biological fluid.

### BRIEF DESCRIPTION OF THE FIGURES

The application file contains at least one photograph executed in color. Copies of this patent application publication with color photographs will be provided by the Office upon request and payment of the necessary fee.

FIG. 1 graphically depicts the profile of the top 20 Aβ proteoforms identified and expressed as a ratio of Aβ1-40. Green color represents Group 1, blue color Group 2, and red Group 3. Aβ1-40 was the most abundant proteoform identified in agreement with previous measures. The next most abundant Aβ31-38 spreads from 25% to 80% of Aβ1-40 abundance.

FIG. 2A is a scatter plot for the three Groups 1, 2, and 3 showing novel Aβ proteoforms accurately discriminates between groups.

FIG. 2B is a scatter plot for the three Groups 1, 2, and 3 showing novel Aβ proteoforms accurately discriminates between amyloid status.

FIG. 3 is a scatter plot of the Aβ proteoform ratios with p-values significantly discriminating PET− from PET+amyloid statuses. Aβ1-43 ratios with other proteoforms separated amyloid negative from amyloid positive subjects.

FIG. 4 graphically depicts Aβ1-43/Aβ1-40 performances as a biomarker for AD diagnosis. Aβ1-43/Aβ31-40 decreased with amyloid-biomarker positivity with CDR0 groups, then decreased further with disease progression (left). It decreased for amyloid-biomarker PET positive compared to PET negative individuals (middle). Receiver operating characteristic curve shows AUC of 0.86 and ability to diagnose amyloid PET status with accuracy of 86% (right).

FIG. 5 is a graph of the profile of the top 20 Aβ proteoforms identified and expressed as a ratio of Aβ1-40. Green color represents Group 1, blue color Group 2, and red Group 3. Aβ1-40 was the most abundant proteoform identified in agreement with previous measures. The next most abundant Aβ1-38 spreads from 30% to 70% of Aβ1-40 abundance.

FIG. 6 is a scatter plot of CSF Aβ42/Aβ40 and Aβ42/Aβ28 to brain amyloid PET status. The three groups represent CDR 0, amyloid-biomarker negative (A); CDR 0, amyloid-biomarker positive (B), and CDR 0.5 amyloid-biomarker positive (C). Error bars are 95% confidence intervals for the mean. The Kruskal-Wallis non-parametric ANOVA separated groups A vs B for both Aβ42/Aβ40 and Aβ42/Aβ28, as well as groups A vs C. Separation between groups B and C was not significant for both Aβ42/Aβ40 and Aβ42/Aβ28.

FIG. 7 graphically depicts the receiver operating characteristic analysis of the various groups compared. The area under the curves (AUC) with 95% confidence intervals. The AUC was 0.88 for Aβ31-42/Aβ31-40 and 0.81 for Aβ31-28/Aβ31-42 in Group A vs B; 0.84 Aβ31-42/Aβ31-40 and 0.82 for Aβ31-28/Aβ31-42 for A vs C. There were no good separators for B vs C, as AUC for Aβ31-42/Aβ31-40 and Aβ1-20/Aβ31-42 were 0.450 and 0.565, respectively.

FIG. 8 graphically depicts the SILK curve of six Ab proteoforms of Aβ1-43, Aβ1-42, Aβ1-40, Aβ1-39, Aβ11-38 and Aβ1-37 of an amyloid-biomarker positive and amyloid-biomarker negative subject. For both, all proteoforms peaked at the same time except for Aβ1-43, indicating equal turnover rated. Aβ1-43 peaked earlier for biomarker positive subjects.

FIG. 9 graphically depict the isotopic enrichment ratios for Aβ1-38/Aβ1-40 displaying both amyloid groups on the same plot (blue open circles, amyloid negative; blue filled circles, amyloid positive) demonstrate similar rates of CSF Aβ1-38/Aβ1-40 turnover regardless of amyloid status. Isotopic enrichment ratios for Aβ1-42/Aβ1-40 (red open triangle, amyloid negative; red filled triangle, amyloid positive) highlight the slightly faster Aβ1-42 turnover kinetics in the amyloid-positive group.

FIG. 10 graphically depict Aβ43, Aβ40, Aβ42 peak areas and SILK curves.

FIG. 11 depicts an illustration of the correlation among biomarkers. The numbers in the lower triangle are used for coloring the corresponding cells in the upper triangle. The blank cells are for insignificant pairs at significance level of 0.05.

FIGS. 12A and 12B depicts illustrations of Abeta 42/40 by MC and disease status. FIG. 12A shows a cross-sectional analysis showing Aβ42/40 vs. EYO by mutation and disease (Baseline, N=462). FIG. 12B shows a longitudinal analysis showing Aβ42/40 vs. EYO by mutation and disease (Longitudinal, N=872).

FIG. 13 depicts an illustration of Abeta 38/40 by MC and disease status.

FIG. 14A-14I depict graphs of Abeta isoforms by EYO (MC and NC). FIG. 14A shows Aβ38/40 vs. EYO. FIG. 14B shows Abeta40, Abeta 42, Abeta37, Abeta38, Abeta39, and Abeta43 vs. EYO in separate graphs. FIG. 14C shows Abeta 42, Abeta37, Abeta38, Abeta39, and Abeta43 vs. EYO in a single merged graph. This data was not broken down by disease status as the sample size of NC CDR>0 was too small. FIG. 14D shows the break down by mutation type for all participants. FIG. 14E shows the break down by mutation type for the mutation carriers. FIG. 14F shows a merged graph of mutation type. FIG. 14G shows a breakdown by mutation position for all participants. FIG. 14H shows a breakdown by mutation position for mutation carriers only. FIG. 14I shows a merged graph of mutation position.

FIG. 15 depicts a graph of Abeta mid-domain peptide by EYO (MC and NC).

FIG. 16 depict graphs showing the correlation of each Abeta isoform with pTau181 (MC and NC). FIG. 16A shows graphs correlating Abeta 40, Abeta42, Abeta37, Abeta38, Abeta39, and Abeta43 with CSF pTau181 for both MC and NC. FIG. 16B shows graphs correlating each Abeta isoform with pTau181 for mutation carrier data only, colored by disease status.

FIG. 17 shows the correlation of each Abeta isoform with pTau217 (MC and NC). FIG. 17A shows graphs correlating Abeta 40, Abeta42, Abeta37, Abeta38, Abeta39, and Abeta43 with CSF pTau217 for both MC and NC. FIG. 17B shows graphs correlating each Abeta isoform with pTau217 for mutation carrier data only, colored by disease status.

FIG. 18 shows a sensitivity/specificity plot for Abeta isoforms.

FIG. 19 shows an illustration of the correlation of Abeta isoforms with CDR by mutation. Note, there are only a few symptomatic non-carriers.

FIG. 20 shows an illustration of the correlation of Abeta isoforms with PET positivity.

FIG. 21 shows an illustration of the correlation of Abeta isoforms with mutation.

FIG. 22A-C show graphs illustrating the association of Abeta isoforms with VILIP (FIG. 22A). SNAP 25 (FIG. 22B), and YKL40 (FIG. 22C).

FIG. 23 shows an illustration of the correlation of Abeta isoforms with age.

### DETAILED DESCRIPTION

Amyloid-beta (Aβ) exists as a plurality of peptides in blood and CSF. Detection and quantification of various Aβ proteoforms in these biological samples has been hampered due to the very low abundance of these polypeptides. The methods disclosed herein employ unique combinations of processing steps that transform a biological sample into a sample suitable for quantifying various Aβ proteoforms. For instance, in some methods of the present disclosure, the processing steps enrich for a plurality of Aβ proteoforms and do not require enzymatic digestion of the Aβ polypeptides prior to analysis. Also described herein are uses of Aβ proteoforms to screen subjects at risk for Alzheimer's disease (AD), stage and/or track progression of AD in a subject; determine the amyloid status of a subject; and treating a subject for AD. For instance, Abeta43 may be used as a stage specific biomarker, as Abeta43 is elevated when the estimated years to onset are less than or equal to 10 (e.g. before amyloid plaques), normalizes during the amyloid plaque stage, and then increases again during the symptomatic stage. These and other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

An antibody, as used herein, refers to a complete antibody as understood in the art, i.e., consisting of two heavy chains and two light chains, and also to any antibody-like molecule that has an antigen binding region, including, but not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. Pat. No. 7,939,313, herein incorporated by reference in its entirety.

The term “Aβ” (also referred to as Abeta or Aβ) refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβmay be attributable to any or all of the above noted forms, as well as to others not described specifically. For example, two such Aβ proteoforms include Aβ40 and Aβ342; with the Aβ42 proteoform being particularly fibrillogenic or insoluble and associated with disease states. The term “Aβ” when used without reference to a specific amino acid sequence typically refers to a plurality of Aβ proteoforms without discrimination among individual Aβ proteoforms. The term “proteoforms” refer to the different forms of a protein produced from the genome and is present in a variety of sequence variations (e.g., amino acid sequence lengths) Specific Aβ proteoforms are identified by the size of the peptide, e.g., Aβ31-42, Aβ31-40, Aβ31-38, Aβ31-43, Aβ37-33, Aβ11-38, etc., where the first integer references the amino terminal amino acid which runs consecutively to the carboxyl terminal amino acid designated by the second integer with reference to DAEFRHDSGYEVHHQKLVFFAEDVGSNKGAIIGLMVGGVVIAT (SEQ ID NO: 1). As used herein, the term “Aβx-42” or “Aβx-40” refers to a plurality of Aβ proteoforms which are greater than 5 amino acids in length include a carboxyl terminal amino acid at position 42 or 40, respectively with reference to SEQ ID NO:1.

As described herein, a ratio calculated from the concentration of one Aβ proteoform in a sample obtained from a subject and compared to the concentration of another Aβ proteoform in the same sample. For example, the term “Aβ31-42/Aβ11-40 value” means the ratio of the amount of Aβ1-42 in a sample obtained from a subject compared to the amount of Aβ1-40 in the same sample. Likewise, the term “Aβ31-43/Aβ31-40 value” means the ratio of the amount of Aβ1-43 in a sample obtained from a subject compared to the amount of Aβ1-40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβamyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [11 C]PIB-PET imaging with mean cortical binding potential (MCBP) score>0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβdeposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ339, Aβ340, and/or total Aβ), as described in U.S. patent Ser. Nos. 14/366,831, 14/523,148, 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβamyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as in individual for whom amyloid imaging may be clinically warranted. As a non-limiting example, a candidate for amyloid imaging may be a subject with one or more clinical signs of Aβ amyloidosis, one or more Aβ plaque associated symptoms, on one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “healthy control group,” “normal group” or a sample from a “healthy” subject means a subject, or group subjects, who is/are diagnosed by a physician as not suffering from Aβ amyloidosis, or a clinical disease associated with Aβ amyloidosis (including but not limited to Alzheimer's disease) based on qualitative or quantitative test results. A “normal” subject is usually about the same age as the individual to be evaluated, including, but not limited, subjects of the same age and subjects within a range of 5 to 10 years.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein variants, arising due alternative splicing of mRNA encoding the protein, post-translational modification of the protein, proteolytic processing of the protein, genetic variations and somatic recombination. The terms “isoform” and “variant” are used interchangeably.

“Significantly deviate from the mean” refers to values that are at least 1 standard deviation, preferably at least 1.3 standard deviations, more preferably at least 1.5 standard deviations or even more preferably at least 2 standard deviations, above or below the mean.

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

The phrase “Aβ therapies” collectively refers to any imaging agent or therapeutic agent contemplated for, or used with, subjects at risk of developing Aβ amyloidosis or AD, subjects diagnosed as having Aβ amyloidosis, or subjects diagnosed as having AD.

### II. Methods for Measuring Aβ Proteoforms

Methods of the present disclose comprise providing an isolated Aβ sample obtained from a subject and measuring one or more Aβ proteoforms.

(a) isolated Aβ sample

An isolated Aβ sample, as used herein, refers to a composition comprising Aβ, wherein Aβ proteoforms have been purified from blood or cerebrospinal fluid (CSF) obtained from a subject. A subject is a mammal, preferably a human. CSF may be obtained by lumbar puncture with or without an indwelling CSF catheter. Multiple blood or CSF samples contemporaneously collected from the subject may be pooled. Blood may be collected by veni-puncture with or without an intravenous catheter, or by a finger stick (or the equivalent thereof). Once collected, blood or CSF samples may be processed according to methods known in the art (e.g., centrifugation to remove whole cells and cellular debris, use of additives designed to stabilize and preserve the specimen prior to analytical testing, etc.). Blood or CSF samples may be used immediately or may be frozen and stored indefinitely.

In isolated Aβ samples of the present disclosure, Aβ proteoforms have been either partially or completely purified from blood or CSF. Methods for purifying Aβ from blood or CSF are known in the art and include, but are not limited to, selective precipitation, size-exclusion chromatography, ion-exchange chromatography, and affinity purification. Suitable methods concentrate a plurality of Aβ proteoforms from blood or CSF.

In an exemplary embodiment, isolated Aβ samples of the present disclosure comprise AD proteoforms that have been purified from blood or CSF by affinity purification. Affinity purification refers to methods that purify a protein of interest by virtue of its specific binding properties to an immobilized ligand. Typically, an immobilized ligand is a ligand attached to a solid support, such as a bead, resin, tissue culture plate, etc. Isolating Aβ proteoforms by affinity purification comprises contacting a sample comprising Aβ with a suitable immobilized ligand and one or more wash steps. Suitable ligands specifically bind Aβ. In one example, a suitable ligand may bind an epitope within the mid domain of Aβ, preferably within amino acids 17 to 28. In another example, a suitable ligand may bind an epitope within the N-terminus of Aβ, preferably within amino acids 1 to 20 of AD. In another example, a suitable ligand may bind an epitope within the C-terminus of Aβ, preferably within amino acids 32 to 43. In still further embodiments, Aβ may be affinity purified from blood or CSF using two or more immobilized ligands either simultaneously or sequentially. In one example, an immobilized ligand binds an epitope within the N-terminus of Aβ and another immobilized ligand binds an epitope within the mid domain of Aβ. In another example, an immobilized ligand binds an epitope within the C-terminus of Aβ and another immobilized ligand binds an epitope within the mid domain of Aβ. In another example, an immobilized ligand binds an epitope within the C-terminus of Aβ and another immobilized ligand binds an epitope within the N-terminus of Aβ. In each of the above embodiments, the epitope binding agent may comprise an antibody or an aptamer. In some embodiments, the epitope-binding agent that specifically binds to amyloid beta is HJ5.1 (mid-domain 17-28), or is an epitope-binding agent that binds the same epitope as HJ5.1 and/or competitively inhibits HJ5.1. In some embodiments, the epitope-binding agent that specifically binds to amyloid beta is HJ3.4 (N-terminal 1-20), or is an epitope-binding agent that binds the same epitope as HJ3.4 and/or competitively inhibits HJ3.4.

An isolated Aβ sample, as used herein, where Aβ proteoforms have been purified by affinity purification, the Aβ proteoforms can be further separated from the immobilized ligand by elution to obtain a supernatant. In some embodiments, purified Aβ proteoforms are further separated from the immobilized ligands using undiluted formic acid. In some embodiments, the supernatant is removed from the sample by drying to obtain a dry isolated Aβ sample. Suitable methods for removing the supernant by drying include but are not limited to centrifugal vacuum concentrators. In one example, the supernant is dried using CentriVap without heat. An isolated Aβ sample (wet or dry) may be used immediately or may be stored indefinitely by methods known in the art.

Prior to analysis, the dry isolated AD sample is reconstituted in a suitable buffer. A suitable buffer allows the Aβ proteoforms to be solubilized in a total volume of 10-50 μL. In an example, a suitable buffer is a mixture of 10% v/v formic acid and 10% v/v acetonitrile.

In various embodiments, methods disclosed herein do not require cleaving purified Aβ proteoforms with a protease. Standard affinity purification protocols in the art, typically require protease digestion of the purified peptides after eluting from the immobilized ligand or while the peptide is bound. Following proteolytic cleavage, the resultant cleavage product are then typically desalted by solid phase extraction prior to detection of the peptide fragments. As noted above, in various embodiments of the present disclosure, the Aβ proteoforms are not cleaved prior to detection.

The biological sample, suitable internal standards, Aβ proteoforms, and mass spectrometry are described in more detail below.

### (b) Biological Sample

Suitable biological samples include a blood sample or a cerebrospinal fluid (CSF) sample obtained from a subject. In some embodiments, the subject is a human. A human subject may be waiting for medical care or treatment, may be under medical care or treatment, or may have received medical care or treatment. In various embodiments, a human subject may be a healthy subject, a subject at risk of developing a neurodegenerative disease, a subject with signs and/or symptoms of a neurodegenerative disease, or a subject diagnosed with a neurodegenerative disease. In further embodiments, the subject may be a candidate for amyloid imaging and/or have a clinical sign of Aβ amyloidosis and/or have an Aβ plaque associated symptom and/or a CAA associated symptom. In other embodiments, the subject is a laboratory animal. In a further embodiment, the subject is a laboratory animal genetically engineered to express human Aβ and optionally one or more additional human protein (e.g., human tau, human ApoE, etc.).

CSF may have been obtained by lumbar puncture with or without an indwelling CSF catheter. Multiple blood or CSF samples contemporaneously collected from the subject may be pooled. Blood may have been collected by veni-puncture with or without an intravenous catheter, or by a finger stick (or the equivalent thereof). Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely. Prior to use in the methods disclosed herein, the biological sample may also have been modified, if needed or desired, to include protease inhibitors, isotope labeled internal standards, detergent(s) and chaotropic agent(s), and/or to deplete other analytes (e.g. proteins peptides, metabolites).

The size of the sample used can and will vary depending upon the sample type, the health status of the subject from whom the sample was obtained, and the analytes to be analyzed (in addition to Aβ). CSF samples volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL.

### (c) Isotope-Labeled, Internal Aβ Standard

Isotope-labeled Aβ may be used as an internal standard to account for variability throughout sample processing and optionally to calculate an absolute concentration. Generally, an isotope-labeled, internal AD standard is added before significant sample processing, and it can be added more than once if needed. See, for instance, the methods described in the Examples below.

Multiple isotope-labeled internal Aβ standards are described herein. All have a heavy isotope label incorporated into at least one amino acid residue. One or more full-length isoforms may be used. Alternatively, or in addition, Aβ isoforms with post-translational modifications and/or peptide fragments of Aβ may also be used, as is known in the art. Generally speaking, the labeled amino acid residues that are incorporated should increase the mass of the peptide without affecting its chemical properties, and the mass shift resulting from the presence of the isotope labels must be sufficient to allow the mass spectrometry method to distinguish the internal standard (IS) from endogenous Aβ analyte signals. As shown herein, suitable heavy isotope labels include, but are not limited to ²H, ¹³C, and ¹⁵N. Typically, about 5-10 ng of internal standard is usually sufficient.

### (d) Aβ Proteoforms

Methods of the present disclosure provide means to measure the various Aβ proteoforms present in a biological sample. In some embodiments, methods herein comprise measuring one or more Aβ proteoforms chosen from Aβ31-40, Aβ31-38, Aβ1-37, Aβ 1-34, Aβ31-39, Aβ33-39, Aβ 1-33, Aβ11-40, Aβ3-40, Aβ1-42, Aβ31-19, Aβ31-25, Aβ1-30, Aβ 1-28, Aβ2-38, Aβ3-38, Aβ3-34, Aβ 11-30, Aβ 11-33, Aβ 11-37, Aβ2-40, Aβ5-40, Aβ11-38, Aβ11-42, Aβ11-34, Aβ7-33, and Aβ1-36. In some embodiments, methods herein comprise measuring one or more Aβ proteoforms chosen from Aβ31-43, Aβ31-25, Aβ7-33, Aβ 1-40, Aβ11-38, Aβ11-42, Aβ11-30, Aβ 1-37, Aβ31-28, Aβ3-40, Aβ31-39, Aβ1-38 and Aβ2-40. In other embodiments, methods herein comprise measuring one or more Aβ proteoforms chosen Aβ31-43, Aβ1-25, and Aβ7-33. In other embodiments, methods herein comprise measuring one or more Aβ proteoforms chosen Aβ1-43, Aβ1-25, Aβ2-4, Aβ31-37, Aβ11-38, and Aβ11-42. In other embodiments, methods herein comprise measuring one or more Aβ proteoforms chosen Aβ1-42, Aβ31-40, and Aβ31-28.

### (e) LC-MS

Another step of the methods disclosed herein comprises performing liquid chromatography—mass spectrometry (LC-MS) with a sample comprising Aβ proteoforms to detect and measure the concentration of at least one Aβ proteoform. Thus, in practice, the disclosed methods use one or more Aβ proteoform to detect and measure the amount of Aβ proteoform present in the biological sample.

Aβ proteoforms may be separated by a liquid chromatography system interfaced with a high-resolution mass spectrometer. Suitable LC-MS systems may comprise a<1.0 mm ID column and use a flow rate less than about 100 μl/min. In preferred embodiments, a nanoflow LC-MS system is used (e.g., about 50-150 μm ID column and a flow rate of <1 μL/min, preferably about 100-1000 nL/min, more preferably about 200-600 nL/min). In an exemplary embodiment, an LC-MS system may comprise a 0.100 mM ID column and use a flow rate of about 400 nL/min.

Tandem mass spectrometry may be used to improve resolution, as is known in the art, or technology may improve to achieve the resolution of tandem mass spectrometry with a single mass analyzer. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer, Orbitrap Fusion™ Lumos™ Mass Spectrometer, Orbitrap Tribrid™ Eclipse™ Mass Spectrometer, Q Exactive Mass Spectrometer, each from ThermoFisher Scientific). In an exemplary embodiment, an LC-MS system may comprise a mass spectrometer selected from Orbitrap Fusion™ Tribrid™ Mass Spectrometer, Orbitrap Fusion™ Lumos™ Mass Spectrometer, Orbitrap Tribrid™ Eclipse™ Mass Spectrometer, or a mass spectrometer with similar or improved ion-focusing and ion-transparency at the quadrupole. Suitable mass spectrometry protocols may be developed by optimizing the number of ions collected prior to analysis (e.g., AGC setting using an orbitrap) and/or injection time. In an exemplary embodiment, a mass spectrometry protocol outlined in the Examples is used.

The present disclosure further contemplates in each of the above methods determining the presence/absence of one or more protein in the biological sample and/or measuring the concentration of one or more additional protein in the biological sample. Alternatively, or in addition, Aβ, ApoE, or any other protein of interest may be identified and/or quantified either by processing a portion of the biological sample in parallel from the biological sample prior to the methods disclosed herein, or from the biological sample during the sample processing steps disclosed herein.

### III. Uses of Aβ Proteoform Measurements

The present disclosure also encompasses the use of measurements of Aβ proteoforms, in blood or CSF as biomarkers of pathological features and/or clinical symptoms of AD in order to diagnose, stage, choose treatments appropriate for a given disease stage, and modify a given treatment regimen (e.g., change a dose, switch to a different drug or treatment modality, etc.). The pathological feature may be an aspect of AD pathology (e.g., presence or amount of AD deposition). Alternatively, or in addition to Ab deposition, a pathological feature may be Aβ-independent. The clinical symptom may be dementia, as measured by a clinically validated instrument (e.g., MMSE, CDR-SB, etc.), or any other clinical symptom associated with AD.

One aspect of the present disclosure encompasses methods to diagnose subjects as having a high risk of conversion to mild cognitive impairment due to Alzheimer's disease. Mild cognitive impairment (MCI) due to Alzheimer's disease (AD) refers to the symptomatic predementia phase of AD. This degree of cognitive impairment is not normal for age and, thus, constructs such as age-associated memory impairment and age-associated cognitive decline do not apply. MCI due to AD is a clinical diagnosis, and clinical criteria for the diagnosis of MCI due to AD are known in the art. See, for instance, Albert et al. Alzheimer's & Dementia, 2011, 7(3): 270-279. Cognitive testing is optimal for objectively assessing the degree of cognitive impairment for a subject. Scores on cognitive tests for subjects with MCI are typically 1 to 1.5 standard deviations below the mean for their age and education matched peers on culturally appropriate normative data (i.e., for the impaired domain(s), when available). The designation of MCI is often supported by a global rating of 0.5 on the Clinical Dementia Rating (CDR) scale. The CDR is a numeric scale used to quantify the severity of symptoms of dementia. Other suitable cognitive tests are known in the art. While suitable tests exist to assess the severity of cognitive impairment, there is a need in the art for a test that identifies subjects with a high degree of confidence years before the onset of MCI due to AD.

In one embodiment, a method to diagnose a subject as having a high risk of conversion to MCI due to AD may comprise (a) providing an isolated Aβ sample obtained from a subject and measuring, in the isolated Aβ sample, one or more AD proteoforms chosen from Aβ1-43, and Aβ1-25, and optionally Aβ1-40 and/or Aβ1-42; and (b) diagnosing the subject as having a high risk of conversion to MCI due to AD when the measured amount significantly deviate from the mean in a control population without brain amyloid plaques as measured by PET imaging and/or Aβx-42/x-40 measurement in CSF. In another embodiment, a method to diagnose a subject as having a high risk of conversion to MCI due to AD may comprise (a) providing an isolated Aβ sample obtained from a subject and measuring, in the isolated Aβ sample, one or more Aβ proteoforms chosen from Aβ1-43, and Aβ1-25, and optionally Aβ1-40 and/or Aβ31-42; and (b) diagnosing the subject as having a high risk of conversion to MCI due to AD when the measured amount significantly deviate from the mean in a control population with a CDR score of 0 and with brain amyloid plaques as measured by PET imaging and/or Aβx-42/x-40 measurement in CSF. In another embodiment, a method to diagnose a subject as having a high risk of conversion to MCI due to AD may comprise (a) providing a first and a second isolated Aβ sample obtained from a subject and measuring, in each isolated Aβ sample, one or more Aβ proteoforms chosen from Aβ1-43, Aβ1-25, and optionally Aβ1-40 and/or Aβ1-42; (b) calculating the change in the amount of each Aβ proteoform measured; and (c) diagnosing the subject as having a high risk of conversion to MCI due to AD when the calculated change(s) significantly deviate from the mean in a control population without brain amyloid plaques as measured by PET imaging and/or Aβx-42/x-40 measurement in CSF or from the mean in a control population with a CDR score of 0 and with brain amyloid plaques as measured by PET imaging and/or Aβx-42/x-40 measurement in CSF.

Another aspect of the present disclosure encompasses methods to detect Aβ amyloidosis in a subject. Generally speaking, the method may comprise (a) providing an isolated Aβ sample obtained from a subject and measuring, in the isolated Aβ sample, one or more Aβ proteoforms chosen from Aβ1-43, Aβ1-25, Aβ7-33, Aβ11-38, Aβ11-42, Aβ1-37, Aβ32-40, Aβ33-40, Aβ11-30, Aβ1-28, and optionally Aβ1-40 and/or Aβ1-42; and (b) detecting amyloidosis when the measured amount of Aβ proteoform(s) significantly deviate from the mean in a control population without brain amyloid plaques as measured by PET imaging and/or Aβx-42/x-40 measurement in CSF. In another embodiment, a method to detect Aβ amyloidosis in a subject may comprise (a) providing a first and a second isolated Aβ sample obtained from a subject and measuring, in each isolated Aβ sample, one or more Aβ proteoforms chosen from Aβ1-43, Aβ1-25, Aβ37-33, Aβ11-38, Aβ11-42, Aβ31-37, Aβ2-40, Aβ3-40, Aβ11-30, Aβ1-28, and optionally Aβ1-40 and/or Aβ1-42; (b) calculating the change in the amount of each AD proteoform(s) measured; and (c) detecting amyloidosis when the calculated change(s) significantly deviate from the mean in a control population without brain amyloid plaques as measured by PET imaging and/or Aβx-42/x-40 measurement in CSF.

“Significantly deviate from the mean” refers to values that are at least 1 standard deviation, preferably at least 1.3 standard deviations, more preferably at least 1.5 standard deviations or even more preferably at least 2 standard deviations, above or below the mean (i.e., 1a, 1.3a, 1.56, or 1.56, respectively. In some embodiments, a is the standard deviation defined by the normal distribution measured in a control population without brain amyloid plaques as measured by PET imaging and/or Aβx-42/x-40 measurement in CSF. In another embodiment, a is the standard deviation defined by the normal distribution measured in a control population with a CDR score of 0 with brain amyloid plaques as measured by PET imaging and/or Aβx-42/x-40 measurement in CSF. In addition to using a threshold (e.g. at least 1 standard deviation above or below the mean), in some embodiment the extent of change above or below the mean may be used to diagnose a subject. An isolated Aβ sample can be obtained from a subject that may or may not be asymptomatic. An “asymptomatic subject” refers to a subject that does not show any signs or symptoms of AD. A subject may however exhibit signs or symptoms of AD (e.g., memory loss, misplacing things, changes in mood or behavior, etc.,) but not show sufficient cognitive or functional impairment for a clinical diagnosis of mild cognitive impairment. In further embodiments, a subject may carry one of the gene mutations known to cause dominantly inherited Alzheimer's disease. In alternative embodiments, a subject may not carry a gene mutation known to cause dominantly inherited Alzheimer's disease. Alzheimer's disease that has no specific family link is referred to as sporadic Alzheimer's disease.

Alternatively or in addition to using a measurement of the amount of one or more Aβ proteoforms, in any of the above embodiments, a ratio calculated from the measured amount Aβ proteoform(s), may be used. Both approaches are detailed in the examples. Mathematical operations other than a ratio may also be used. For instance, the examples use Aβ proteoforms values in various statistical models (e.g., linear regressions, etc.) in conjunction with other known biomarkers (e.g. APOE ε4 status, age, sex, cognitive test scores, functional test scores, etc.). Selection of measurements and choice of mathematical operations may be optimized to maximize specificity of the method. For instance, diagnostic accuracy may be evaluated by area under the ROC curve and in some embodiments, an ROC AUC value of 0.7 or greater is set as a threshold (e.g., 0.7, 0.75, 0.8, 0.85, 0.9, 0.95, etc.).

Brain amyloid plaques in humans are routinely measured by amyloid-positron emission tomography (PET). For instance, 11 C-Pittsburgh compound B (PiB) PET imaging of cortical Aβ-plaques is commonly used to detect Aβ-plaque pathology. The standard uptake value ratio (SUVR) of cortical PiB-PET reliably identifies significant cortical Aβ-plaques and is used to classify subjects as PIB positive (SUVR>1.25) or negative (SUVR<1.25). Accordingly, in the above embodiments, a control population without brain amyloid plaques as measured by PET imaging may refer to a population of subjects that have a cortical PiB-PET SUVR<1.25. Other values of PiB binding (e.g., mean cortical binding potential) or analyses of regions of interest other than the cortical region may also be used to classify subjects as PIB positive or negative. Other PET imaging agents may also be used.

A control population without brain amyloid plaques as measured by Aβx-42/x-40 measurement in CSF may refer to a population of subjects that has an Aβx-42/x-40 measurement of <0.12 when measured by mass spectrometry, as described in Patterson et al, Annals of Neurology, 2015. Thus, in contrast, a control population with brain amyloid plaques as measured by Aβx-42/x-40 measurement in CSF may refer to a population of subjects that has an Aβx-42/x-40 measurement of >0.12 when measured by mass spectrometry.

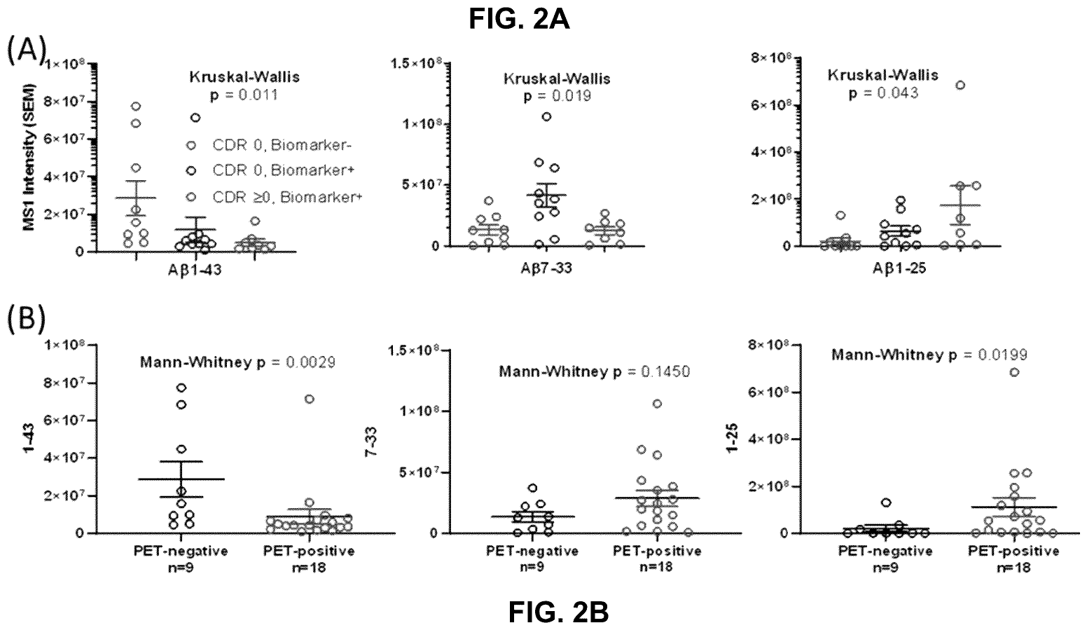

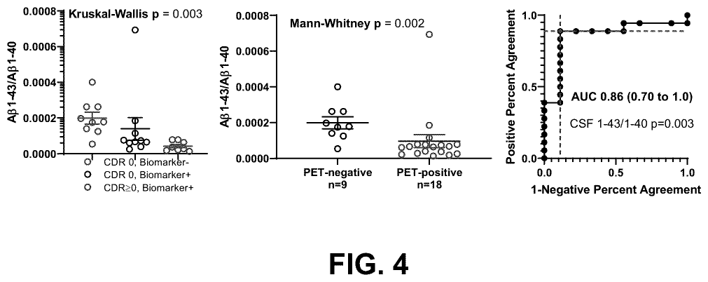

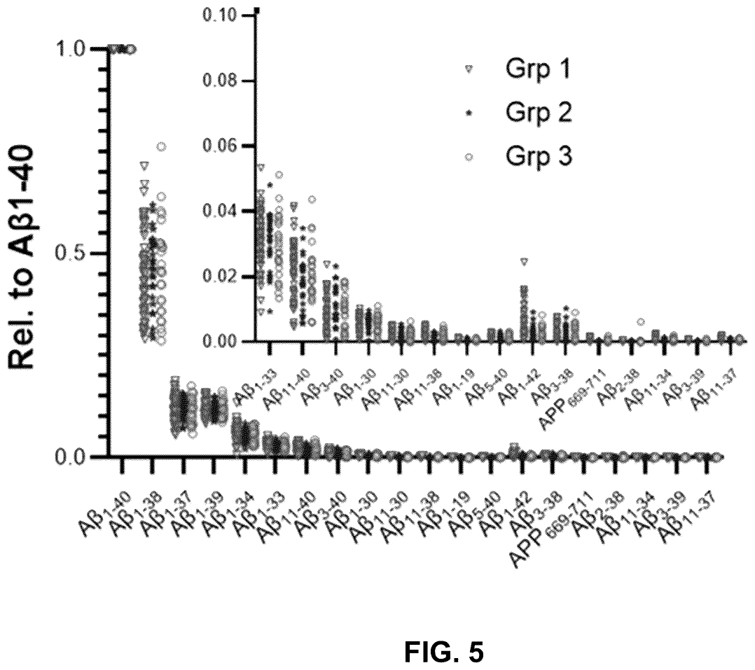

In an exemplary embodiment, a method to diagnose a subject as having a high risk of conversion to MCI due to AD or a subject's stage of AD may comprise (a) providing an isolated Aβ sample obtained from a subject and measuring, in the isolated Aβ sample, one or more AD proteoforms chosen from Aβ1-43, Aβ1-25, and optionally Aβ1-40 and/or Aβ31-42; and (b) diagnosing the subject as having a high risk of conversion to MCI due to AD or staging the subject's AD when the measured amount of Aβ proteoform(s) significantly deviate from the mean in a control population without brain amyloid plaques as measured by PET imaging and/or Aβx-42/x-40 measurement in CSF or when the measured amount of Aβ proteoform(s) significantly deviate from the mean in a control population with a CDR score of 0 and with brain amyloid plaques as measured by PET imaging and/or Aβx-42/x-40 measurement in CSF. FIGS. 2A and 4, illustrates the change in the amount of measured Aβ proteoforms between cognitively normal (CDR=0) and amyloid negative subjects, cognitively normal (CDR=0) and amyloid positive subjects, and very mild dementia (CDR=0.5) and amyloid positive subjects. A decrease in Aβ1-43 levels that significantly deviate from the mean can indicate disease progression to MCI due to AD; and an increase in Aβ31-25 levels that significantly deviate from the mean can indicate disease progression to MCI due to AD; As noted above, additional mathematical operations may be performed with the measurements of Aβ proteoform(s), including but not limited to ratio between a first measured Aβ proteoform and second measured Aβ proteoform. For example, a ratio of Aβ1-43 and Aβ1-40 can be calculated. A decrease in the value of Aβ1-43/Aβ1-40 that significantly deviate from the mean can indicate disease progression to MCI due to AD.

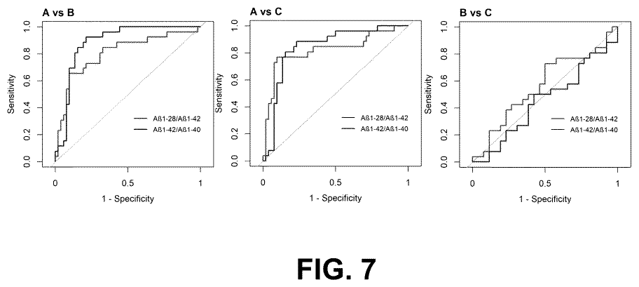

In another exemplary embodiment, methods to detect Aβ amyloidosis in a subject may comprise (a) providing an isolated Aβ sample obtained from a subject and measuring, in the isolated Aβ sample, one or more Aβ proteoforms chosen from Aβ1-43, Aβ1-25, Aβ37-33, Aβ11-38, Aβ11-42, Aβ1-37, Aβ2-40, Aβ3-40, Aβ11-30, Aβ31-28, and optionally Aβ1-40 and/or Aβ1-42; and (b) detecting amyloidosis when the measured amount of Aβ proteoform(s) significantly deviate from the mean in a control population without brain amyloid plaques as measured by PET imaging and/or Aβx-42/x-40 measurement in CSF. FIGS. 2B, 3, 4, 6 and 7 illustrates the change in the amount of measured Aβ proteoforms between cognitively normal (CDR=0) and amyloid negative subjects, cognitively normal (CDR=0) and amyloid positive subjects, and very mild dementia (CDR=0.5) and amyloid positive subjects. A decrease in Aβ31-43 levels that significantly deviate from the mean can indicate an amyloid positive subject; an increase in Aβ1-25 levels can indicate an amyloid positive subject; and an increase in Aβ7-33 levels can indicate an amyloid positive subject. As discussed herein, additional mathematical operations may be performed with the measurements of Aβ proteoform(s), including but not limited to ratio between a first measured Aβ proteoform and second measured Aβ proteoform. For example, a ratio of Aβ1-43 and Aβ1-40; a ratio of Aβ1-42 and Aβ1-40; a ratio of Aβ1-43 and Aβ11-38; a ratio of Aβ1-43 and Aβ11-42, a ratio of Aβ1-37 and Aβ31-43; a ratio of Aβ2-40 and Aβ11-43; and a ratio of Aβ1-42 and Aβ11-28 can be calculated. A decrease in the value of Aβ1-43/Aβ1-40 and/or Aβ1-42/Aβ31-40 and/or Aβx-42/Aβx-40; Aβ1-43/Aβ11-38 and/or Aβ1-42/Aβ1-28 that significantly deviate from the mean can indicate an amyloid positive subject. An increase in the value of Aβ1-37/Aβ31-43 and/or Aβ2-40/Aβ1-43 that significantly deviate from the mean can indicate an amyloid positive subject.

Methods for measuring AD proteoforms are described in Section II, and incorporated into this section by reference. For instance, using the protocol detailed in the below Examples. A skilled artisan will appreciate, however, that the absolute value may vary depending upon the protocol and the source/specifications of internal standards used for absolute quantitation.

In a preferred embodiment, an isolated Aβ sample comprises Aβ proteoform(s) that have been purified from blood or CSF by affinity purification Aβ proteoform(s) concentration is measured by mass spectrometry. In an exemplary embodiment, a mass spectrometry protocol outlined in the Examples is used.

In a specific embodiment, the present disclosure provides a method for measuring Alzheimer disease (AD)—related pathology in a subject, the method comprising providing an isolated Aβ sample obtained from a subject and measuring, in the isolated Aβ sample, one or more Aβ proteoforms, wherein the amount of the quantified Aβ proteoforms, or their ratios, is a representation of AD-related pathology in a brain of a subject.

In another specific embodiment, the present disclosure provides a method for determining a subject's amyloid status, the method comprising providing an isolated Aβ sample obtained from a subject and measuring, in the isolated Aβ sample, one or more Aβ proteoforms, wherein the amount of the quantified Aβ proteoforms, or their ratios, is a representation of AD-related amyloid beta deposition in a brain of a subject and predicts amyloid-positivity as determined by PIB-PET, for instance by PiB-PET SUVR as described in Ann Neurol 2016; 80:379-387.

In another specific embodiment, the present disclosure provides a method for diagnosing Alzheimer's disease, the method comprising providing an isolated Aβ sample obtained from a subject and measuring, in the isolated Aβ sample, one or more Aβ proteoforms and diagnosing Alzheimer's disease when the amount of the quantified Aβ proteoforms, or their ratios differs by about 1.5—or more, where—is the standard deviation defined by the normal distribution measured in a control population does not have clinical signs or symptoms of a AD and that is amyloid negative as measured by PET imaging (for instance by PiB-PET SUVR as described in Ann Neurol 2016; 80:379-387) and/or Aβ42/40 measurement in CSF (for instance, a cutoff value for CSF Aβ42/40 calculated from PiB-PET SUVR (Ann Neurol2016; 80:379-387) that maximizes sensitivity %+Specificity %).

In another specific embodiment, the present disclosure provides a method for measuring Alzheimer disease (AD) progression in a subject, the method comprising providing a first CSF or blood sample and a second CSF or blood sample, wherein each sample is obtained from a single subject, and each sample is isolated for Aβ; and for each sample, measuring one or more Aβ proteoforms; and calculating the difference between the quantified Aβ proteoforms in the second sample and the first sample, wherein the amount of the quantified Aβ proteoforms, or their ratios is a statistically significant increase or decrease in the quantified Aβ proteoform in the second sample indicates progression of the subject's Alzheimer's disease.

### IV. Methods of Treatment

Another aspect of the present disclosure is a method for treating a subject in need thereof. The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. In some embodiments, a subject receiving treatment is asymptomatic. An “asymptomatic subject,” as used herein, refers to a subject that does not show any signs or symptoms of AD. In other embodiments, a subject may exhibit signs or symptoms of AD (e.g., memory loss, misplacing things, changes in mood or behavior, etc.,) but not show sufficient cognitive or functional impairment for a clinical diagnosis of mild cognitive impairment due to Alzheimer's disease. The phrase “mild cognitive impairment due to Alzheimer's disease” is defined in Section I. A symptomatic or an asymptomatic subject may have Aβ amyloidosis; however, prior knowledge of Aβ amyloidosis is not a requisite for treatment. In still further embodiments, a subject may be diagnosed as having AD. In any of the aforementioned embodiments, a subject may carry one of the gene mutations known to cause dominantly inherited Alzheimer's disease. In alternative embodiments, a subject may not carry a gene mutation known to cause dominantly inherited Alzheimer's disease.

In one embodiment, a method for treating a subject as described above may comprise (a) providing an isolated Aβ sample obtained from a subject and measuring, in the isolated Aβ sample, one or more Aβ proteoform(s) chosen from Aβ1-43, Aβ1-25, Aβ37-33, Aβ11-38, Aβ11-42, Aβ31-37, Aβ2-40, Aβ33-40, Aβ11-30, Aβ31-28, and optionally Aβ1-40 and/or Aβ1-42; and (b) administering a pharmaceutical composition to the subject when the measured Aβ proteoform level(s) significantly deviate from the mean in a control population without brain amyloid plaques as measured by PET imaging and/or Aβ42/40 measurement in CSF. In another embodiment, a method for treating a subject as described above may comprise (a) providing an isolated Aβ sample obtained from a subject and measuring, in the isolated Aβ sample, one or more Aβ proteoform(s) chosen from Aβ31-43, Aβ1-25, Aβ37-33, Aβ11-38, Aβ11-42, Aβ1-37, Aβ32-40, Aβ33-40, Aβ11-30, Aβ1-28, and optionally Aβ1-40 and/or Aβ1-42; and; (b) calculating the change in the Aβ proteoform level(s); and (c) administering a pharmaceutical composition to the subject when the calculated change(s) significantly deviate from the mean in a control population without brain amyloid plaques as measured by PET imaging and/or Aβ42/40 measurement in CSF. “Significantly deviate from the mean” refers to values that are at least 1 standard deviation, preferably at least 1.3 standard deviations, more preferably at least 1.5 standard deviations or even more preferably at least 2 standard deviations, above or below the mean (i.e., 1a, 1.36, 1.56, or 1.56, respectively, where a is the standard deviation defined by the normal distribution measured in a control population without brain amyloid plaques as measured by PET imaging and/or Aβ42/40 measurement in CSF). In addition to using a threshold (e.g. at least 1 standard deviation above or below the mean), in some embodiment the extent of change above or below the mean may be used as criteria for treating a subject.

Alternatively or in addition to using a measurement of Aβ proteoform level(s), in any of the above embodiments, a ratio calculated from the measured Aβ proteoform level(s), may be used. A ratio calculated from the measured Aβ proteoform level(s) may be a ratio Aβ1-43 and Aβ1-40; a ratio of Aβ1-42 and Aβ1-40; a ratio of Aβ1-43 and Aβ11-38; a ratio of Aβ1-43 and Aβ11-42, a ratio of Aβ1-37 and Aβ1-43; a ratio of Aβ2-40 and Aβ1-43; a ratio between Aβx-42 and Aβx-40; and a ratio of Aβ1-42 and Aβ1-28 can be calculated. Mathematical operations other than a ratio may also be used. For instance, the examples use Aβ proteoform values in various statistical models (e.g., linear regressions, LME curves, LOESS curves, etc.) in conjunction with other known biomarkers (e.g. APOE ε4 status, age, sex, cognitive test scores, functional test scores, etc.).

Many imaging agents and therapeutic agents contemplated for, or used with, subjects at risk of developing Aβ amyloidosis or AD, subjects diagnosed as having Aβ amyloidosis, subjects diagnosed as having a tauopathy, or subjects diagnosed as having AD, target a specific pathophysiological change. For instance, Aβ targeting therapies are generally designed to decrease Aβ production, antagonize Aβ aggregation or increase brain Aβ clearance; tau targeting therapies are generally designed to alter tau phosphorylation patterns, antagonize tau aggregation, or increase NFT clearance; a variety of therapies are designed to reduce CNS inflammation or brain insulin resistance; etc. The efficacy of these various agents can be determined by measuring various Aβ proteoforms using the methods disclosed herein.

In an exemplary embodiment, the efficacy of imaging agents and therapeutic agents contemplated for, or used with, subjects at risk of developing Aβ amyloidosis or AD, subjects diagnosed as having Aβ amyloidosis, subjects diagnosed as having a tauopathy, or subjects diagnosed as having AD (collectively referred to herein as “Aβ and tau therapies”) can be improved by administering the Aβ or tau therapy to subjects that have certain Aβ proteoform levels as measured by methods disclosed herein. For instance, preferred therapeutic agents may include those designed to prevent a subject from becoming amyloid positive (e.g., amyloid targeting therapies designed to decrease Aβ production, antagonize Aβ aggregation, etc.). As another example, preferred therapeutic agents may include those designed to prevent amyloid deposition from increasing or reduce a subject's existing plaque load. As another example, preferred therapeutic agents may include those designed to prevent amyloid deposition from increasing, reduce a subject's existing plaque load, prevent tau aggregation, or target NFTs. As another example, preferred therapeutic agents may include those designed to prevent amyloid deposition from increasing, reduce a subject's existing plaque load, prevent tau aggregation, or target NFTs, as well as those specific for subjects with AD. The details disclosed herein can similarly be used to administer therapeutic agents designed for other targets (e.g., CNS inflammation, ApoE, etc.), including but not limited to those identified in the following paragraphs.

In one example, the present disclosure provides a method for treating a subject having an increased risk of conversion to MCI due to AD or amyloidosis, the method comprising (a) providing an isolated Aβ sample obtained from a subject and measuring Aβ proteoform level(s); and (b) administering a pharmaceutical composition to the subject when AD proteoform level significantly deviates from the mean.

In each of the above embodiments, a pharmaceutical composition may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.)

Alternatively, a pharmaceutical composition may comprise an active pharmaceutical ingredient. Non-limiting examples of active pharmaceutical ingredients include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Ap antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAβ106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1 D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

Methods for measuring Aβ proteoforms are described in Section II, and incorporated into this section by reference. In an exemplary embodiment, a mass spectrometry protocol outlined in the Examples is used.

### V. Clinical Trials

Another aspect of the present disclosure is a method for enrolling a subject into a clinical trial, in particular a clinical trial for an Aβ therapy, provided all other criteria for the clinical trial have been met. In one embodiment, a method for enrolling a subject into a clinical trial may comprise (a) providing an isolated Aβ sample obtained from a subject and measuring, in the isolated Aβ sample, one or more Aβ proteoform(s) chosen from Aβ1-43, Aβ1-25, Aβ37-33, Aβ11-38, Aβ11-42, Aβ1-37, Aβ32-40, Aβ3-40, Aβ11-30, Aβ31-28, and optionally Aβ11-40 and/or Aβ11-42; and (b) enrolling the subject into a clinical trial when the measured Aβ proteoform level(s) significantly deviate from the mean in a control population without brain amyloid plaques as measured by PET imaging and/or Aβ42/40 measurement in CSF. “Significantly deviate from the mean” refers to values that are at least 1 standard deviation, preferably at least 1.3 standard deviations, more preferably at least 1.5 standard deviations or even more preferably at least 2 standard deviations, above or below the mean (i.e., 1σ, 1.3σ, 1.5σ, or 1.5σ, respectively, where a is the standard deviation defined by the normal distribution measured in a control population without brain amyloid plaques as measured by PET imaging and/or Aβ42/40 measurement in CSF). In addition to using a threshold (e.g. at least 1 standard deviation above or below the mean), in some embodiment the extent of change above or below the mean may be used as criteria for enrolling a subject.

Alternatively or in addition to using a measurement of Aβ proteoform level(s), in any of the above embodiments, a ratio calculated from the measured Aβ proteoform level(s), may be used. Mathematical operations other than a ratio may also be used. For instance, the examples use Aβ proteoform values in various statistical models (e.g., linear regressions, LME curves, LOESS curves, etc.) in conjunction with other known biomarkers (e.g. APOE ε4 status, age, sex, cognitive test scores, functional test scores, etc.).

The design of clinical trials for Aβ therapies can be greatly aided by the methods disclosed herein. Many clinical trials are designed to test the efficacy of imaging agents or therapeutic agents that target a specific pathophysiological change which occurs prior to the onset of AD symptoms. Clinical trials enrolling subjects with symptoms of AD (e.g., after the onset of MCI due to AD) would also benefit from being able to accurately stage an enrollee's AD status in order to determine if efficacy is associated with a particular stage of AD. Accordingly, measuring Aβ proteoform level(s) as described herein prior to enrolling a subject in a clinical trial, in particular into a treatment arm of a clinical trial, may result in smaller trials and/or improved outcomes. In some instances, methods described herein may be developed and used as a companion diagnostic for a therapeutic agent.

Methods for measuring Aβ proteoform levels are described in Section II, and incorporated into this section by reference. In an exemplary embodiment, a mass spectrometry protocol outlined in the Examples is used.

In each of the above embodiments, a subject may be enrolled into a treatment arm of the clinical trial. The “treatment” is defined in Section V. Subjects enrolled in the treatment arm of a clinical trial may be administered a pharmaceutical composition. In some embodiments, a pharmaceutical composition may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.). Alternatively, a pharmaceutical composition may comprise an active pharmaceutical ingredient. Non-limiting examples of active pharmaceutical ingredients include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAβ106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A.

In each of the above embodiments, a subject may or may not be symptomatic. An “asymptomatic subject,” as used herein, refers to a subject that does not show any signs or symptoms of AD. Alternatively, a subject may exhibit signs or symptoms of AD (e.g., memory loss, misplacing things, changes in mood or behavior, etc.,) but not show sufficient cognitive or functional impairment for a clinical diagnosis of mild cognitive impairment. A symptomatic or an asymptomatic subject may have Aβ amyloidosis; however, prior knowledge of Aβ amyloidosis is not a requisite for treatment. In still further embodiments, a subject may have AD. In any of the aforementioned embodiments, a subject may carry one of the gene mutations known to cause dominantly inherited Alzheimer's disease. In alternative embodiments, a subject may not carry a gene mutation known to cause dominantly inherited Alzheimer's disease.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### VI. Kits

The current disclosure provides kits for measuring Aβ proteoforms or monitoring the progression or treatment of a neurological or neurodegenerative disease associated with AD. Generally, a kit comprises a regents for generating an isolated Aβ sample, including but not limited to, one or more ligands which specifically bind Aβ, a solid support to immobilize the one or more ligands, labeled amino acid standard, buffers, means for collecting biological samples, and instructions for detecting and measuring the amount of a Aβ proteoform.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1

The pathological process of AD begins decades before cognitive decline. It has become apparent that for disease-modifying therapies to be highly effective, early intervention is required. In order to screen those at risk, improved biomarkers of AD pathology are urgently needed to identify individuals early while damage is not too severe and potentially reversible. Amyloid-beta (Aβ) 40 and 42 measures in blood, CSF and brain are established AD biomarkers. It is thought that longer forms than Aβ42 may also be present in all three specimens, but none have been detected by mass spectrometry to date. Presented here are a new generation of biomarkers which could be at the frontier of screening the at-risk population in order to detect early stages of AD decades before disease onset. These screening biomarker tools could also be used to monitor the effectiveness of disease modifying therapies. A highly sensitive and specific mass spectrometry (MS) method using immunoprecipitation and high resolution mass spectrometry was developed to identify a new generation of AD biomarkers. Aβ variants were detected in the cerebrospinal fluid (CSF) of AD patients and age-matched normal controls, and their turnover rates were compared. Aβ variants across two stages of AD to were analyzed to resolve the metabolic differences of the Aβ variants that are most pathologically relevant to AD. By measuring intact CSF Aβ by mass spectrometry, a better understanding and bridging the information gap regarding full sequence heterogeneity of Aβ in humans is demonstrated in the present example. In this example, the successful measurement of intact Aβ across AD cohorts and identified: i) an array of novel Aβ proteoforms and diagnostic markers to screen those at risk for AD, ii) prognostic markers to track progression within a clinical timeframe, and iii) proteoforms discriminating amyloid plaque status (positive vs. negative) and Clinical Dementia Rating (CDR). The assay described herein provides a faster, more economical, and more specific way to quantify Aβ proteoforms.

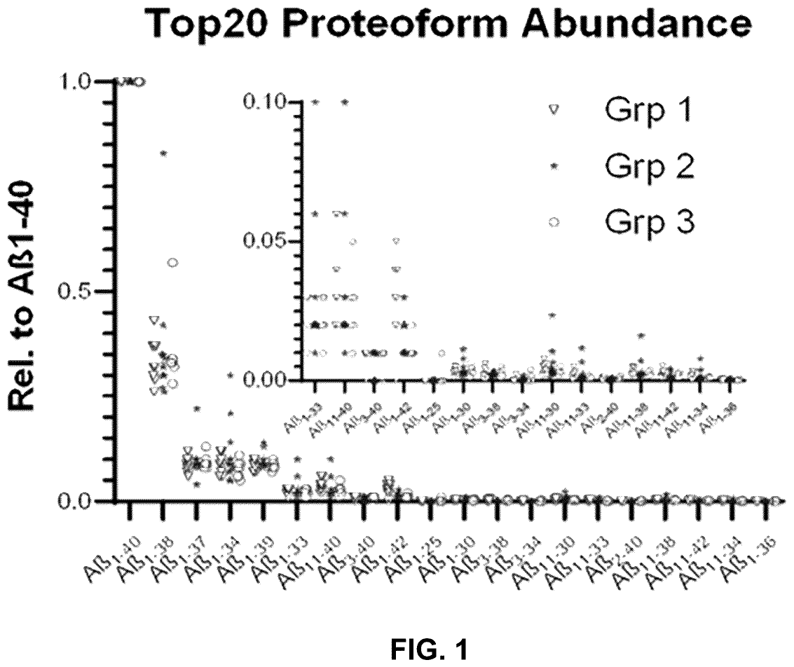

The present example compares Aβ proteoforms present in the CSF of participants who were cognitively normal or had mild cognitive impairment characteristic of symptomatic AD dementia. Group 1 participants (n=9) were cognitively normal (CDR=O) and amyloid negative, Group 2 participants (n=10) were cognitively normal (CDR=O) and amyloid positive, and Group 3 participants (n=8) had very mild dementia (CDR=0.5) and were amyloid positive (n=8). These CSF samples were assayed by immunoprecipitation and mass spectrometry (IPMS) to define the various Aβ proteoform signatures and other post-translational modifications in CSF. Seventy-one diverse Aβ proteoforms were identified. The top 20 verified by accurate mass of intact proteoforms and their corresponding tandem mass spectrometry of fragments generated in the mass spectrometer are shown in FIG. 1. The features of the detected Aβ consist of truncated and shorter forms relative to Aβ1-40 and extensions at both terminal extended forms. Thirty-four Aβ proteoforms were truncated either at the N- and 37 at the C-terminal and 13 at both ends. Four proteoforms were identified with extensions at either end. The shortest proteoform had 19 amino acid residues and the longest had 43. However, only 32 proteoforms were verified to be true identifications with good quality mass spectra. Among these, the top 20 most abundant were normalized with Aβ1-40 abundance due to the relatively wide dynamics in Aβ concentrations observed in CSF. FIG. 1 illustrates the dynamics of CSF Aβ concentrations with a ratio=1 for Aβ1-40 and ˜0.01 for Aβ1-36, which is two orders of magnitude separation. Up to 60% of CSF Aβ represents Aβ31-40, and five Aβ species make up close to 90% of all Aβ. The insert in FIG. 1 shows these species to highlight the least abundant species (a 10× magnification) and reveals the diversity of the other 40% of Aβ proteoforms identified.

Among the novel proteoforms quantified, three less abundant species showed trends of discriminating between groups with significant p-values (FIG. 2A). Aβ31-43 was significant in discriminating Group 1 (CDR0, amyloid negative) from Group 2 (CDR0, amyloid positive) and Group 3 (CDR0.5, amyloid positive) individuals with decreasing abundance of Aβ31-43 from Group 1 to Group 3 (Group 1>Group 2>Group 3). In contrast, Aβ31-25 showed the reverse trend of increasing in concentration as shown with Group 1<Group 2<Group 3, where more Aβ31-25 seems to be generated with disease progression. These two novel proteoforms fully discriminate PET-negative from PET-positive cohorts. However, Aβ37-33 was elevated with biomarker positivity (Group 2>Group 1), but dropped back to lower levels in Group 3 (Group 2>Group 3). This could be prognostic in confidently discriminating Group 2 from Group 1, and Group 2 from Group 3. Altogether, Aβ31-43 levels decrease with disease progression, Aβ31-25 levels increase in CSF with disease progression, and Aβ7-33 levels increase with biomarker positivity but decrease once clinical symptoms appear. These three proteoforms accurately predicted amyloid positivity with the area under the curve (AUC) of receiver operating characteristics to various degrees. Aβ31-43 (AUC=0.85), Aβ31-25 (AUC=0.78) and Aβ37-33 (AUC=0.68) predicted amyloid positivity at least 85%, 78% and 68%, respectively.

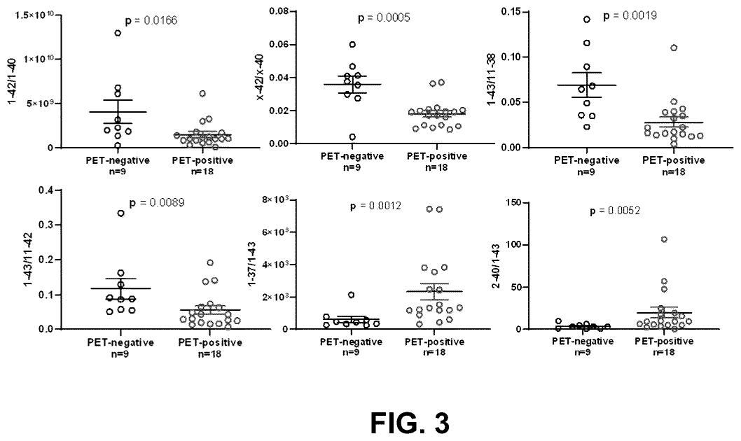

All possible ratios of the 32 proteoforms verified were calculated and the top six reported showed high discriminatory power between amyloid negative and positive participants (FIG. 3). Aβx-42/Aβx-40 has been the established ratio traditionally used to amyloid positive and amyloid negative groups. However, this ratio could be a combination of many N-terminally truncated forms of the two main proteoforms of Aβ31-42 and Aβ31-40, leading to a degree of uncertainty in their diagnostic values. This was exactly the case when these two ratios were used for comparison shown in FIG. 3. The diagnostic ability of Aβ1-43/Aβ1-40 has been demonstrated in FIG. 4, where the ratio separated each group and decreased with disease progression between CDR0 groups, as well as conversion from CDR0 to CDR>0. Receiver operating characteristics curve showed AUC distinguished PET status by 86%. Other significant ratios discriminating amyloid positive from amyloid negative groups were Aβ1-43/Aβ11-38, Aβ31-43/Aβ11-42, Aβ31-37/Aβ1-43 and Aβ32-40/Aβ31-43.

Next, to validate Aβ proteoforms in a larger cohort discriminating CDR status with an independent sample set. The validation cohort of 104 CSF samples was from the Knight ADRC Biomarker Core was analyzed as described herein. This cohort comprised Group A: N=52 PET negative and CDR 0, Group: B N=26 PET positive and CDR 0, and Group C: N=26 PET positive and CDR 0.5.

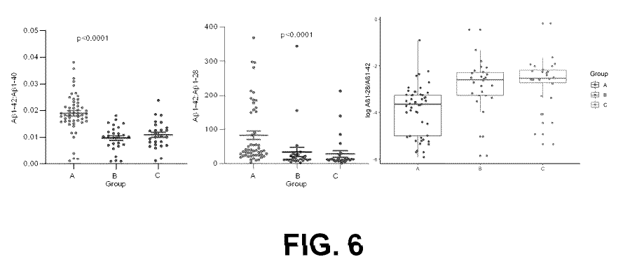

In this cohort, 71 diverse Aβ proteoforms were identified. FIG. 4 illustrates the top 20 most abundant proteoforms. Again, the dynamic range of proteoforms detected was more than two orders of magnitude. Twenty-four manually verified proteoform mass spectra were used to calculate all combinations of proteoform ratios to determine their ability to predict PET positivity. One-way ANOVA with Dunn's multiple comparison statistical analysis for the 3 groups were analyzed. Out of the 112 ratio combinations derived, eight showed statistical significance but two stood out and are shown in FIG. 6. Aβ1-42/Aβ31-40 and Aβ31-42/Aβ31-28 were highly significant in discriminating between Group A vs B. and Group A vs C. Aβ31-42/Aβ31-40 and Aβ31-42/Aβ31-28 decreased in amyloid positive individuals independently with highly significant p-values (p<0.0001). However, Groups B and C could not be separated by these two ratios or a log transformation of Aβ1-28/Aβ1-42. The ability to discriminate groups was further analyzed by receiver operating characteristics of Aβ1-42/Aβ31-40 and Aβ31-28/Aβ31-42. This demonstrated both CSF Aβ31-42/Aβ31-40 and Aβ31-28/Aβ1-42 were biomarker status predictors. The area under the curves (AUC) with 95% confidence intervals were 0.86 for Aβ31-42/Aβ31-40 and 0.81 for Aβ31-28/Aβ31-42 in Group A vs (B,C). On the other hand, the AUC for (A, B) vs C were 0.74 for Aβ1-42/Aβ31-40 and 0.71 for Aβ1-28/Aβ1-42. Group A vs B was 0.88 Aβ1-42/Aβ1-40 and 0.81 for Aβ1-28/Aβ1-42. Group A vs C was 0.84 Aβ1-42/Aβ31-40 and 0.82 for Aβ31-28/Aβ1-42. For Group B vs C, Aβ31-42/Aβ31-40 was not a good separator and Aβ31-28/Aβ31-42 was also not significant in separating the two. However, candidate ratios which could be useful include, but are not limited to Aβ3-40/Aβ11-30 (AUC 0.65), Aβ31-40/Aβ31-28 (AUC 0.64), Aβ33-40/Aβ1-28 (AUC 0.64), and Aβ1-39/Aβ1-28, Aβ11-30/Aβ31-40, Aβ1-37/Aβ31-28, Aβ1-38/Aβ31-28, Aβ11-30/Aβ31-37 all with AUC 0.62.

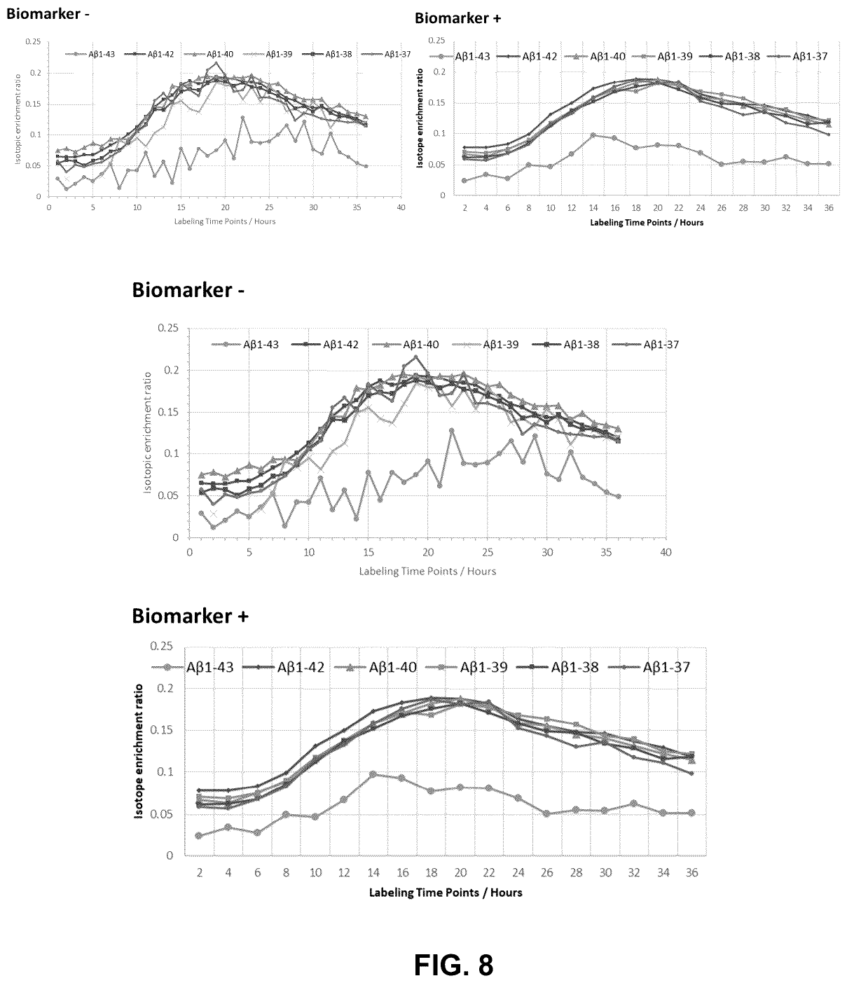

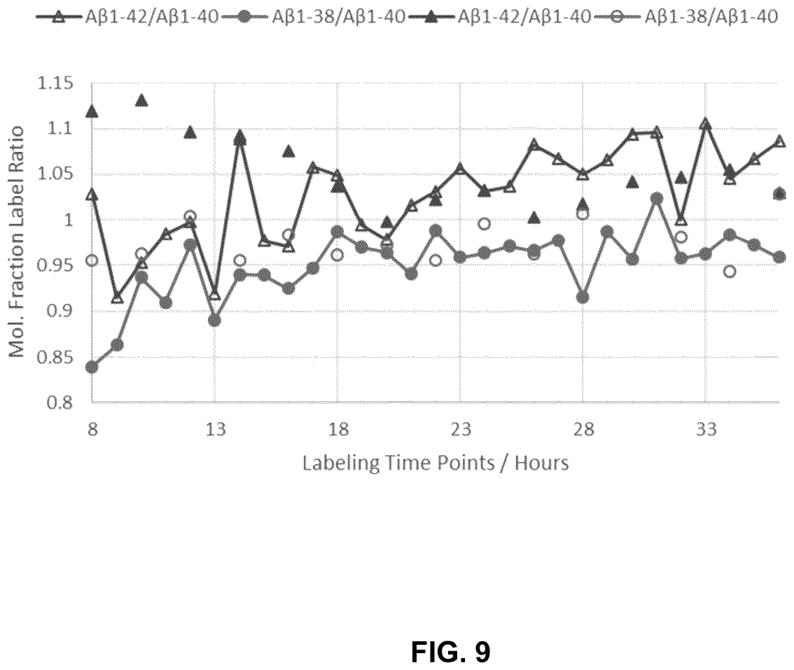

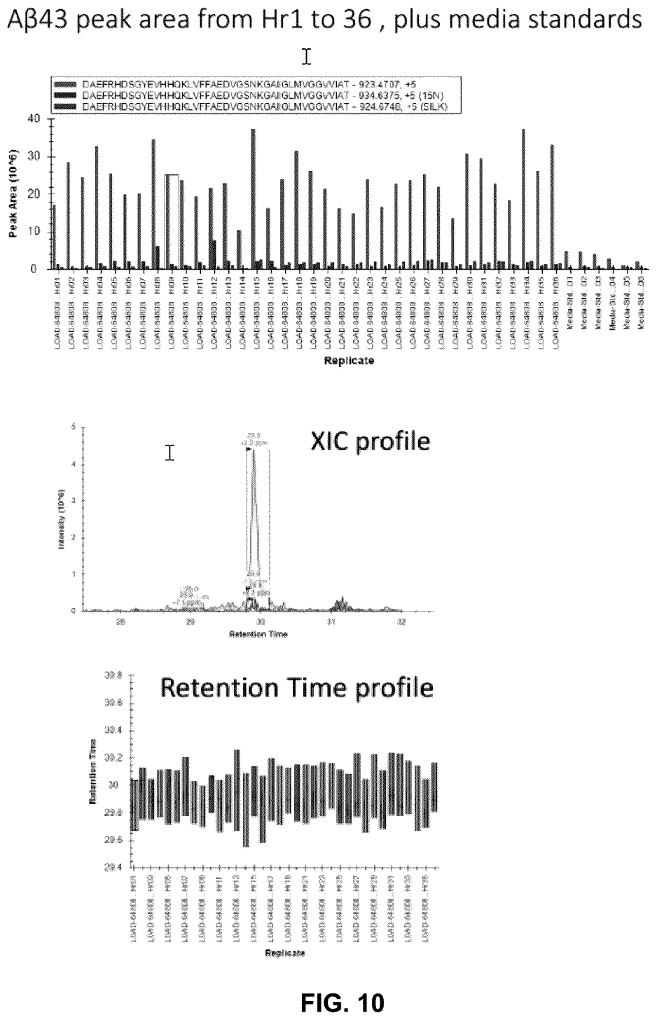

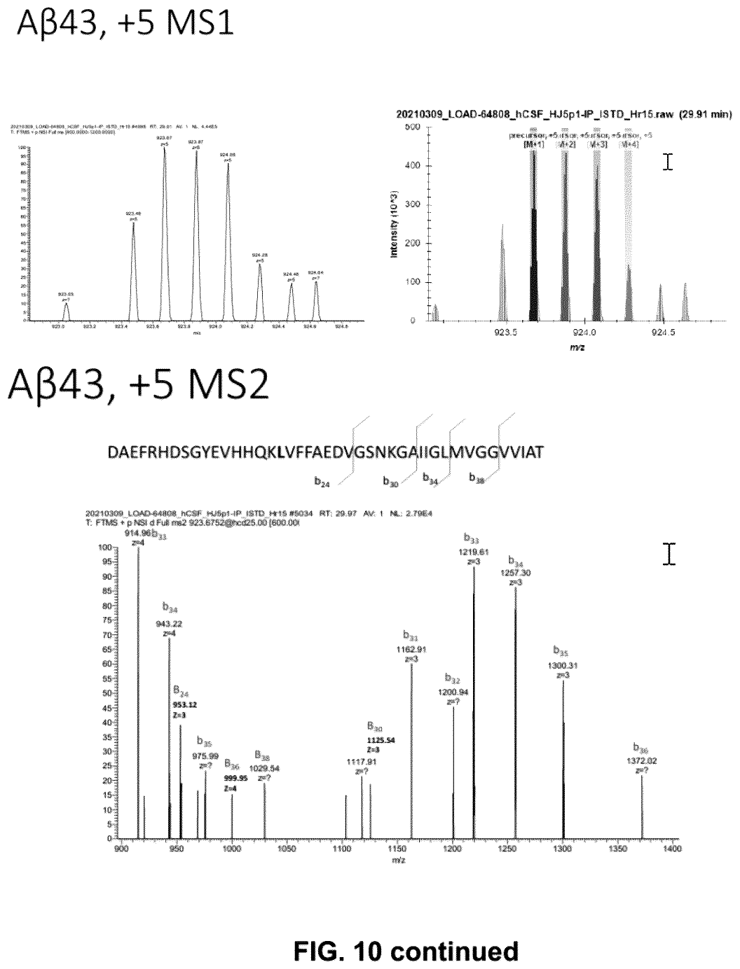

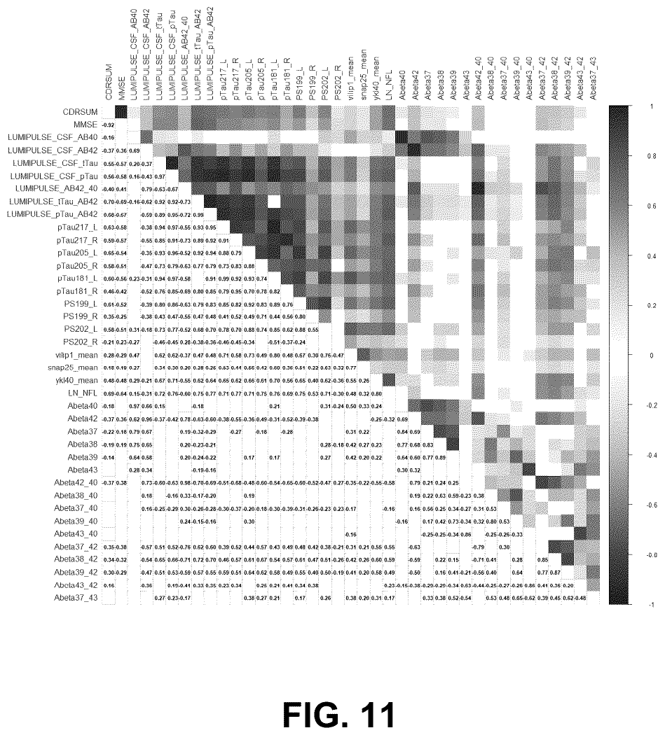

Stable isotope label kinetics (SILK) technology was used in the analysis of intact Aβ proteoforms to understand the kinetics (change in production and clearance over time) of proteoforms most relevant in distinguishing AD cohorts with minimal overlap. Analyses on 0.5 mL aliquots of CSF collected every hour for 36 hours obtained from prior SILK studies conducted in the Bateman lab were performed. Due to sample volume limitations, one participant each of CDR=0 and CDR>0 were selected and analyzed. The CSF was processed by immunoprecipitation (IP) and analyzed using a high-resolution Orbitrap Fusion mass spectrometer (MS), as described herein, the Aβ proteoforms quantified in the study were the canonical Aβ1-37, Aβ31-38, Aβ1-39, Aβ1-40, Aβ31-42 and Aβ31-43 and their corresponding isotopically enriched forms. Isotopic enrichment ratios were calculated and plotted against the CSF time profile to elucidate differences in the kinetics of Aβ proteoforms in CSF for both biomarker negative and biomarker positive individuals (FIG. 8). Aβ31-43 was the least abundant and needed optimization to measure 1-10% of signal accurately to determine meaningful kinetics. For both amyloid-negative and amyloid-positive individuals, all proteoforms labeling kinetics peaked at the same time, indicating equal turnover rates. FIG. 9 compares the ratios of Aβ1-38/Aβ1-40 SILK as an unchanging biomarker to Aβ31-42/Aβ1-40 SILK for both amyloid statuses. As expected, the Aβ11-38/Aβ13-40 SILK were similar over time between amyloid groups (FIG. 9), indicating no difference in kinetic processing between Aβ31-38 and Aβ31-40. In contrast, the CSF Aβ31-42/Aβ1-40 SILK had faster soluble Aβ31-42 turnover kinetics in the amyloid-positive individual (FIG. 9). The Aβ31-42/Aβ31-40 SILK labeling was higher initially in the amyloid positive group until a drop after 16 hours of the amyloid-positive group indicating faster Aβ1-42 turnover and aggregation in those with CNS amyloidosis. This is in agreement with previous findings with earlier mass spectrometry results. It is unknown whether this discriminates pre-clinical and clinical stages of AD.

In conclusion, this example successfully performed whole Aβ proteoform profiling in CSF. Several novel Aβ proteoforms were identified and shown to separate amyloid PET+from PET− individuals. In addition, proteoform ratios were found to distinguish amyloid PET+from PET− individuals. Aβ1-42/Aβ1-40 was found to discriminate PET+from PET− individuals, just as it has been diagnostic in predicting AD pathology in other c-terminal amyloid-beta mass spectrometry based assays. Aβ31-43 ratios with other proteoforms significantly discriminated PET− vs PET+individuals and Aβ31-28/Aβ1-42 was found to be a potential biomarker separating CDR=0 from CDR>0 as well. The kinetics of intact Aβ1-42/Aβ1-40 was demonstrated, as well as the SILK of other novel proteoforms. The present example demonstrates the ability to detect Aβ1-43 proteoform, which has not been detected and measured in CSF by any mass spectrometry based assay. Together with other novel proteoforms identified, Aβ31-43 seems to be a candidate biomarker for which SILK could be explored.

Analytical Method used for CSF intact Aβ IPMS: is as follows. Briefly, Aβ peptides CSF were purified through immunoprecipitation using a mixture of Aβ-specific antibodies HJ5.1 (mid-domain 17-28) and Aβ N-terminal antibody HJ3.4 (N-terminal 1-20) coupled to magnetic Dynabeads M-280 (standard operating protocol attached). In order to ensure reliable quantification, and eliminate batch to batch variations a synthetic Aβ31-34 containing stable isotope of uniformly [15N]label was used as internal standard (rPeptide, CA) and spiked into the CSF sample prior to immunoprecipitation. The samples solubilized in a solution of 10% formic acid/10% acetonitrile was analyzed by LC-MS with Aβ variants separated by M class UHPLC (Waters Corporation) coupled to a Orbitrap Fusion MS (Thermoscientific, San Jose CA). Briefly, liquid chromatography separation of amyloid peptides was performed on HSS T3 column material (75 μm×100 mm) maintained at 65° C. MS analysis of the eluting peptides was carried out in positive mode and in a data-dependent fashion. Data acquisition was performed with 1 μscan/acquisition with the resolution set to 60.000 and AGC target values of 1×10⁶ in MS and 1×10⁴ MS/MS mode. The Precursor isolation width was 2 m/z units, and ions were fragmented by higher-energy collision-induced dissociation (HCD) at a normalized collision energy of 25%. Mass spectrometry data were analyzed using the Skyline software package and exported to MS Excel. The integrated peak areas of precursor [M+1]to [M+4]were summed. GraphPad Prizm was used for further data and statistical analyses. Analysis of variance (ANOVA, with Kruskal-Wallis test). A p value threshold of 0.01 was used for assessment of the statistical significance.

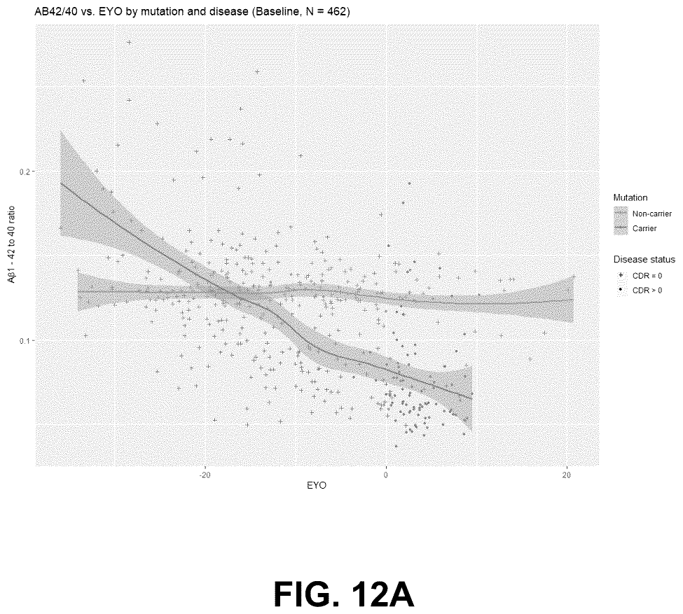

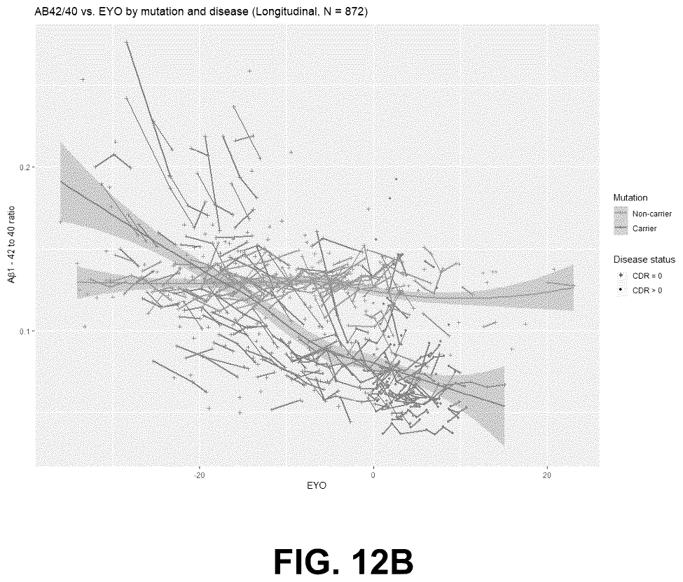

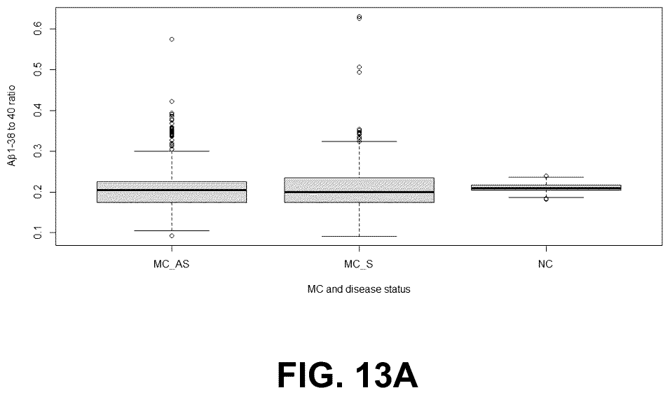

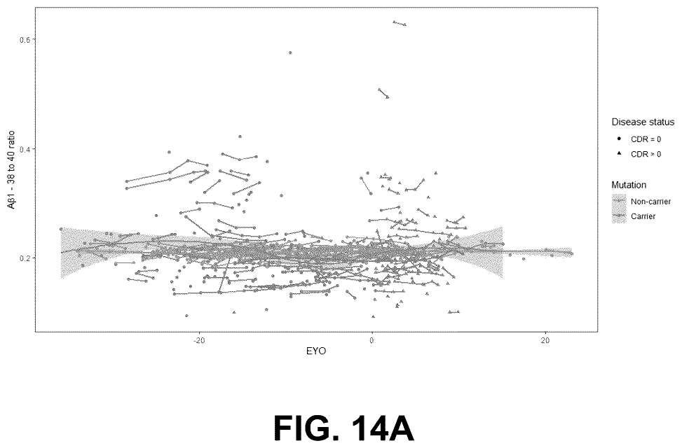

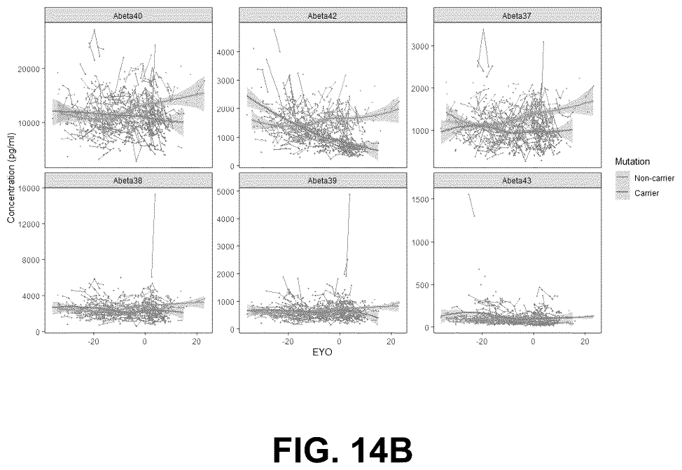

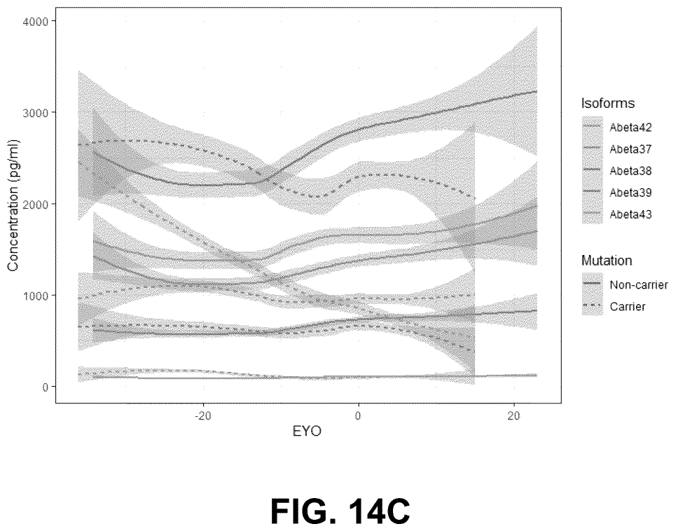

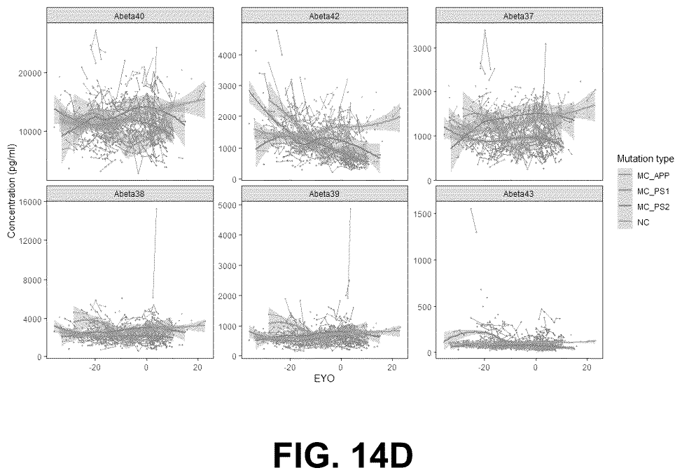

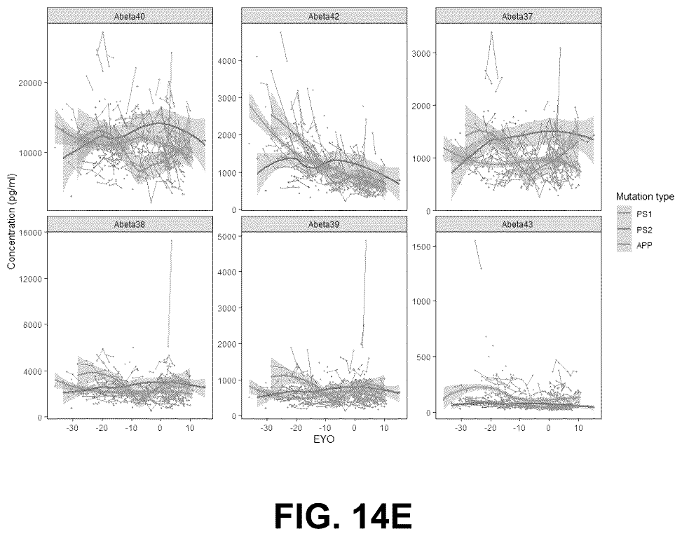

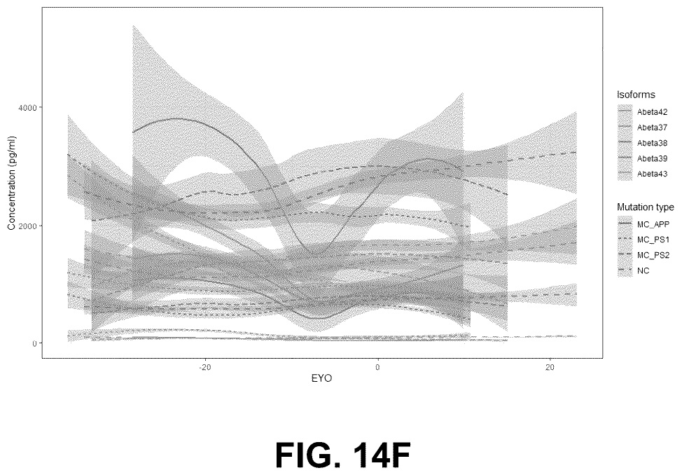

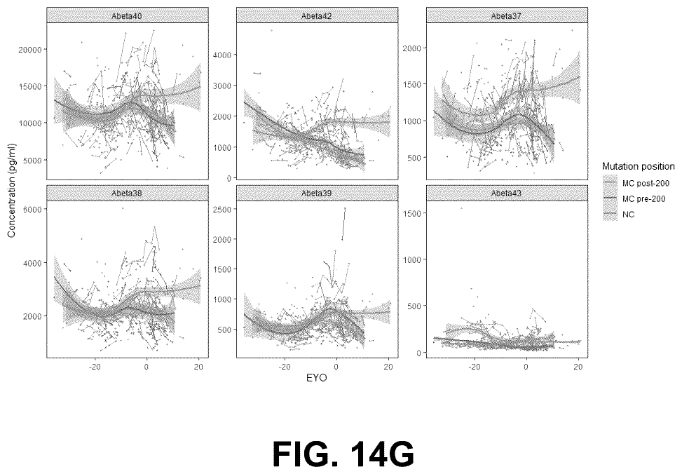

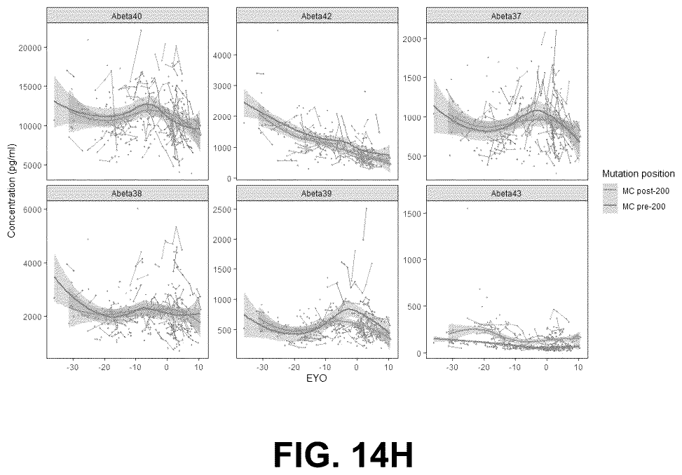

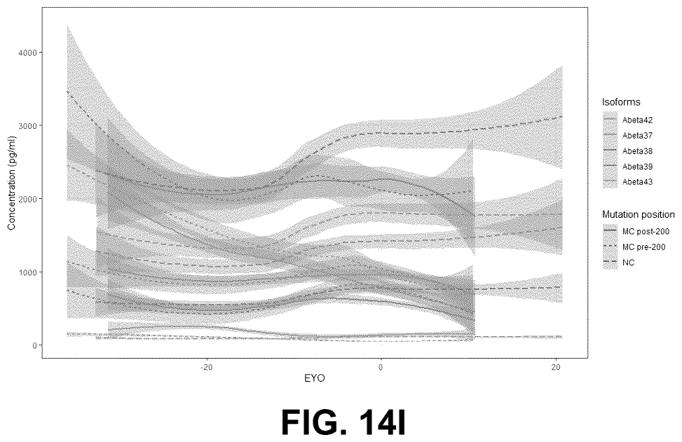

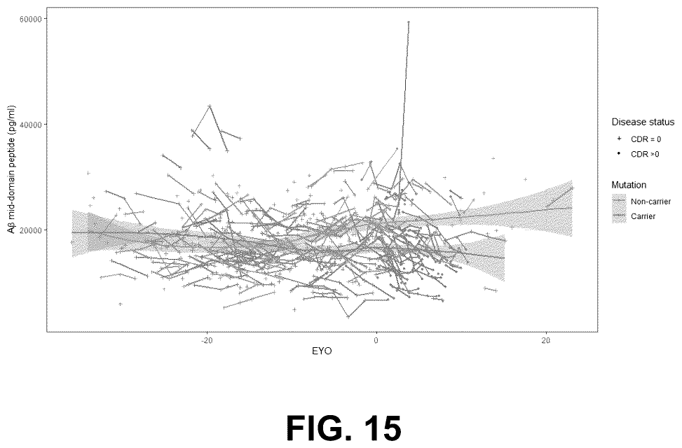

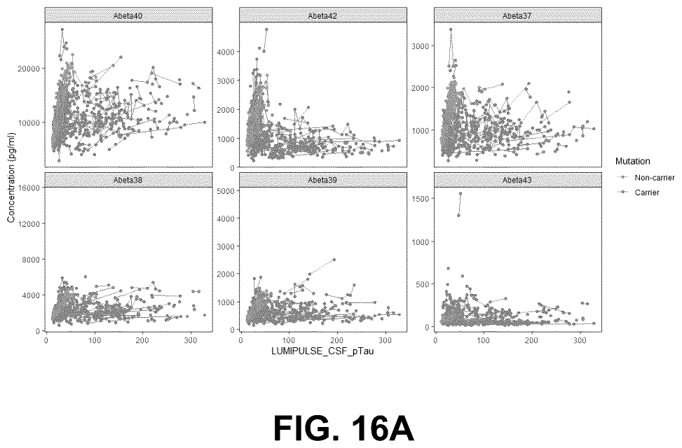

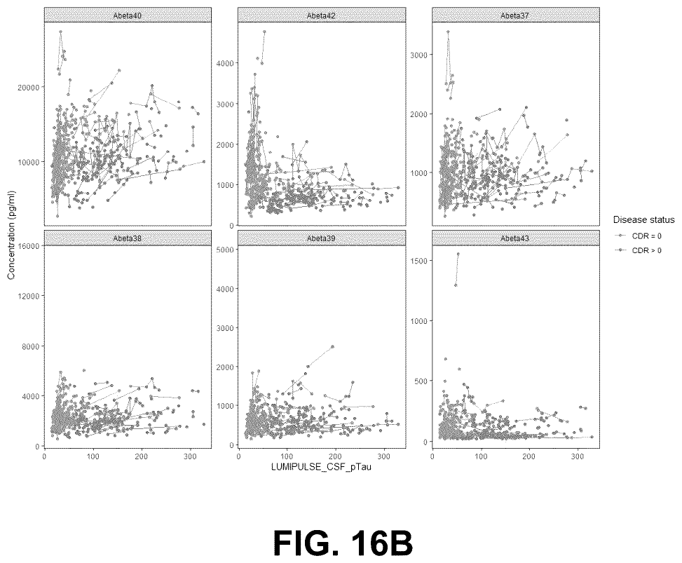

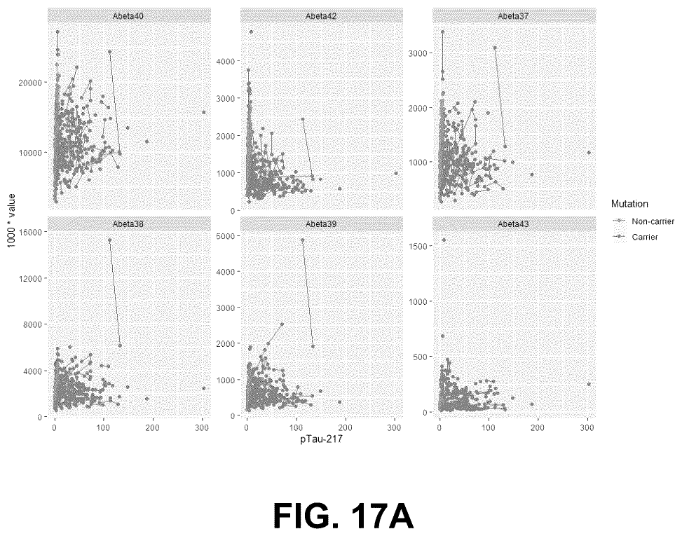

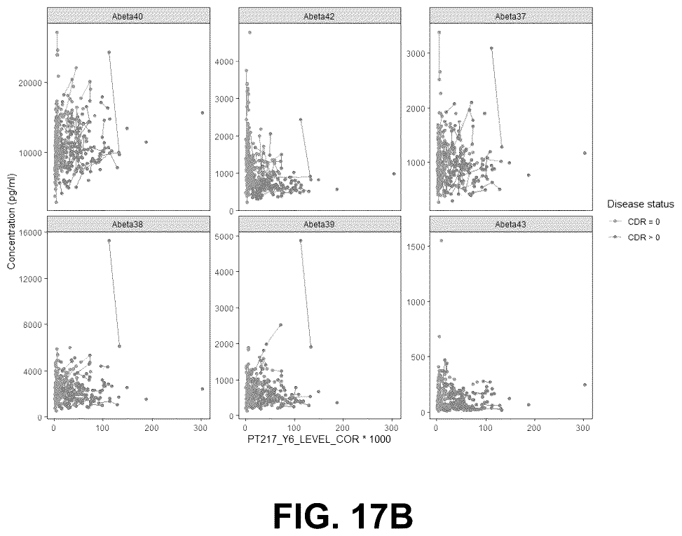

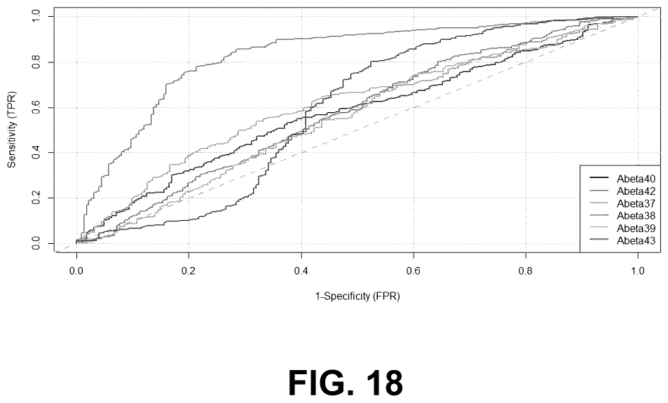

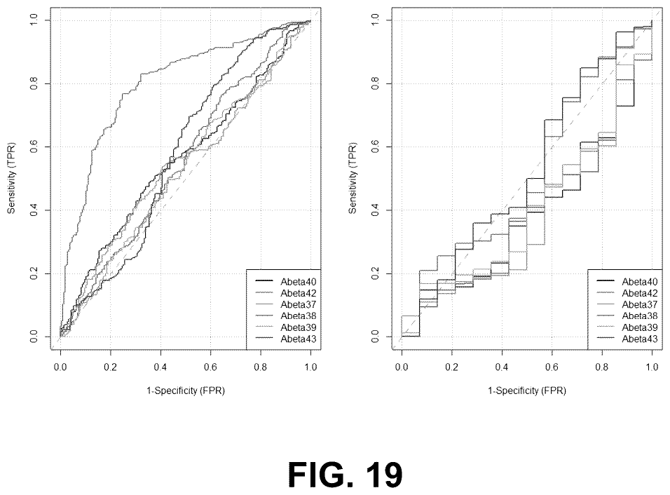

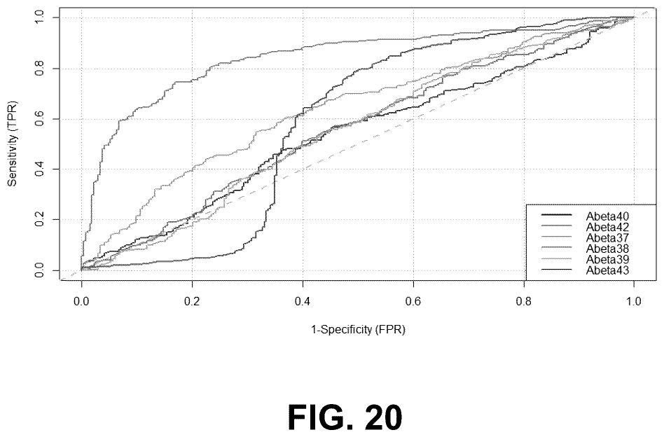

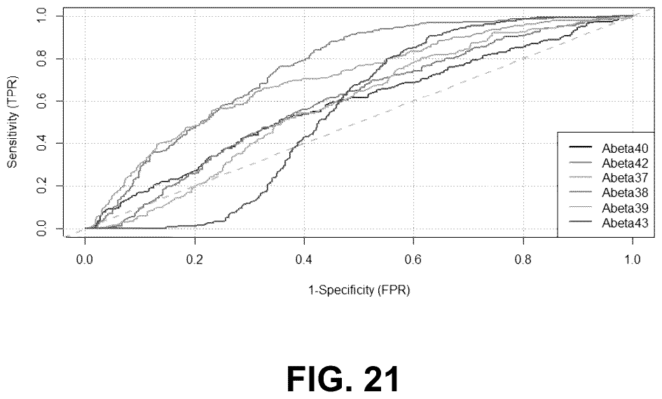

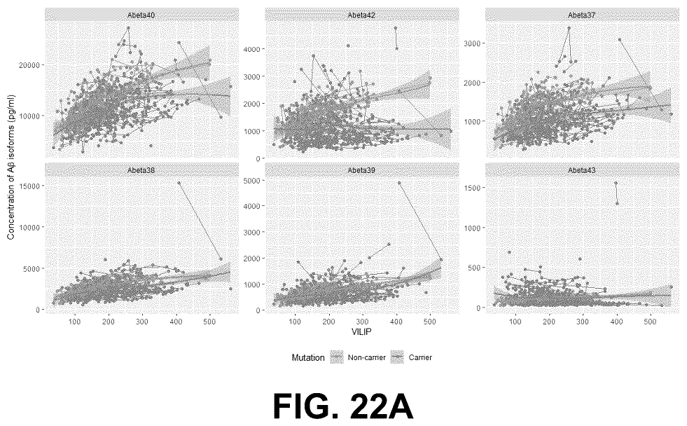

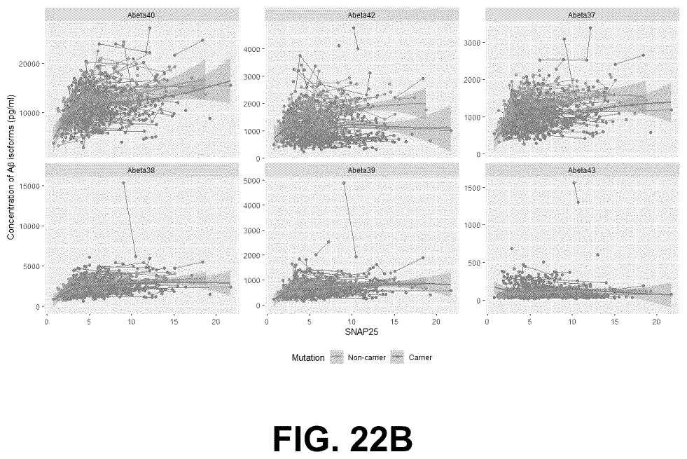

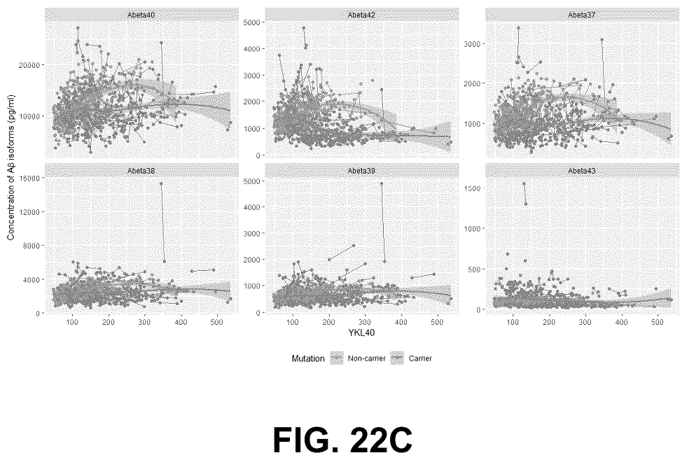

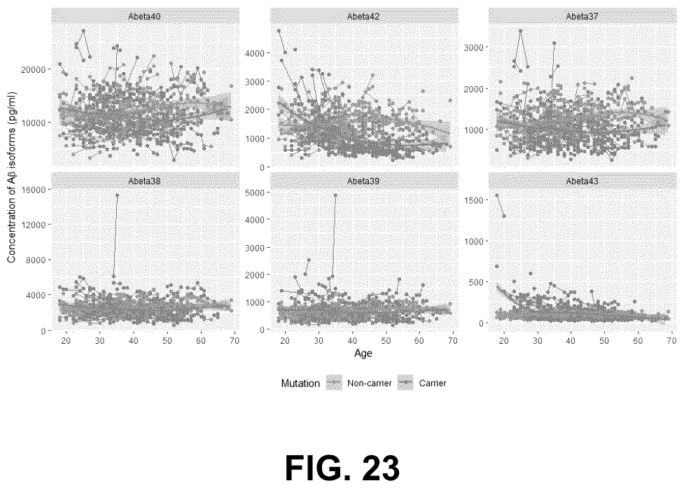

TABLE 1
(see FIG. 11-23)

| | | | Pre- | | | | | | | |
|---|---|---|---|---|---|---|---|---|---|---|
| | | | symptomatic | | Symptomatic | | | | | |
| | Non-carrier | | carrier | | carrier | | Overall | | | P-value |
| | (N = 183) | | (N = 190) | | (N = 92) | | (N = 465) | | P-value | (adjusted) |
| Sex | | | | | | | | | | |
| Male | 74 | (40.4%) | 84 | (44.2%) | 42 | (45.7%) | 200 | (43.0%) | 0.6479 | 0.5804 |
| Female | 109 | (59.6%) | 106 | (55.8%) | 50 | (54.3%) | 265 | (57.0%) | | |
| Ethnicity | | | | | | | | | | |
| Non-hispanic | 167 | (91.3%) | 167 | (87.9%) | 73 | (79.3%) | 407 | (87.5%) | 0.0526 | 0.6296 |
| Hispanic | 15 | (8.2%) | 23 | (12.1%) | 18 | (19.6%) | 56 | (12.0%) | | |
| Unknown | 1 | (0.5%) | 0 | (0%) | 1 | (1.1%) | 2 | (0.4%) | | |
| Education | 14.96 | (2.63) | 14.98 | (2.87) | 13.61 | (3.45) | 14.70 | (2.95) | 4e−04 | 0.0053 |
| (years) | | | | | | | | | | |
| Number of | 0.55 | (1.26) | 0.49 | (1.15) | 0.36 | (0.74) | 0.49 | (1.13) | 0.4284 | 0.3036 |
| visits | | | | | | | | | | |
| CDR-SB | 0.06 | (0.28) | 0.04 | (0.14) | 3.65 | (3.54) | 0.76 | (2.13) | <1e−04 | <.0001 |
| MMSE | 28.96 | (1.33) | 29.02 | (1.25) | 22.87 | (6.63) | 27.78 | (3.98) | <1e−04 | <.0001 |
| Age of onset | 48.31 | (7.09) | 48.54 | (6.83) | 43.14 | (8.11) | 47.38 | (7.49) | <1e−04 | <.0001 |
| Apoe e4 | | | | | | | | | 0.5095 | 0.7259 |
| Non-carrier | 120 | (65.6%) | 135 | (71.1%) | 64 | (69.6%) | 319 | (68.6%) | | |
| Carrier | 63 | (34.4%) | 55 | (28.9%) | 28 | (30.4%) | 146 | (31.4%) | | |
| Amyloid PET | | | | | | | | | <1e−04 | <.0001 |
| status | | | | | | | | | | |
| Positive | 2 | (1.1%) | 76 | (40.0%) | 57 | (62.0%) | 135 | (29.0%) | | |
| (PiB >1.4) | | | | | | | | | | |
| Negative | 159 | (86.9%) | 89 | (46.8%) | 5 | (5.4%) | 253 | (54.4%) | | |
| (PiB <1.4) | | | | | | | | | | |
| PIB SUVR | 1.06 | (0.17) | 1.62 | (0.79) | 2.83 | (1.28) | 1.58 | (0.95) | <1e−04 | <.0001 |
| Hippocampus | 8820.22 | (774.68) | 8889.15 | (991.66) | 7278.59 | (1290.12) | 8576.19 | (1144.27) | <1e−04 | <.0001 |
| volume | | | | | | | | | | |
| Lumipulse | | | | | | | | | | |
| CSF (pg/ml) | | | | | | | | | | |
| Abeta1-40 | 8811.64 | (2801.08) | 8467.12 | (2942.68) | 8008.39 | (2630.65) | 8511.30 | (2837.03) | 0.0828 | 0.0686 |
| Abeta1-42 | 798.47 | (284.25) | 676.72 | (361.22) | 391.61 | (202.55) | 667.92 | (338.79) | <1e−04 | <.0001 |
| Total tau | 267.53 | (113.06) | 370.65 | (237.13) | 744.68 | (378.67) | 408.48 | (297.85) | <1e−04 | <.0001 |
| p-tau 181 | 28.91 | (14.16) | 49.38 | (44.85) | 122.19 | (69.01) | 55.98 | (54.99) | <1e−04 | <.0001 |
| p-tau 217 | 4.00 | (3.78) | 14.01 | (17.51) | 53.44 | (46.13) | 17.89 | (29.76) | <1e−04 | <.0001 |
| Abeta1-42/40 | 0.09 | (0.01) | 0.08 | (0.03) | 0.05 | (0.02) | 0.08 | (0.03) | <1e−04 | <.0001 |
| Total tau/ | 0.36 | (0.27) | 0.73 | (0.68) | 2.35 | (1.34) | 0.92 | (1.07) | <1e−04 | <.0001 |
| Abeta1-42 | | | | | | | | | | |
| p-tau 181/ | 0.04 | (0.04) | 0.10 | (0.13) | 0.39 | (0.25) | 0.13 | (0.19) | <1e−04 | <.0001 |
| Abeta1-42 | | | | | | | | | | |
| VILIP-1 | 150.47 | (62.05) | 172.05 | (69.41) | 227.33 | (98.91) | 174.55 | (78.63) | <1e−04 | <.0001 |
| SNAP25 | 4.91 | (2.86) | 5.30 | (2.85) | 6.69 | (3.47) | 5.43 | (3.05) | <1e−04 | <.0001 |
| YKL-40 | 130.54 | (57.84) | 132.07 | (58.24) | 232.30 | (90.66) | 152.06 | (77.52) | <1e−04 | <.0001 |
| log(NFL) | 5.36 | (0.55) | 5.46 | (0.53) | 6.65 | (0.70) | 5.65 | (0.76) | <1e−04 | <.0001 |
| C2N CSF | | | | | | | | | | |
| (ng/ml) | | | | | | | | | | |
| Abeta40 | 12.25 | (3.49) | 11.56 | (3.70) | 10.80 | (3.21) | 11.68 | (3.56) | 0.005 | 0.0028 |
| Abeta42 | 1.55 | (0.48) | 1.36 | (0.70) | 0.82 | (0.39) | 1.33 | (0.63) | <1e−04 | <.0001 |
| Abeta37 | 1.28 | (0.37) | 1.01 | (0.41) | 0.93 | (0.35) | 1.10 | (0.41) | <1e−04 | <.0001 |
| Abeta38 | 2.56 | (0.74) | 2.41 | (0.98) | 2.27 | (1.00) | 2.44 | (0.90) | 0.0317 | 0.0074 |
| Abeta39 | 0.66 | (0.21) | 0.63 | (0.30) | 0.61 | (0.33) | 0.64 | (0.27) | 0.3042 | 0.2540 |
| Abeta43 | 0.09 | (0.03) | 0.12 | (0.14) | 0.11 | (0.09) | 0.11 | (0.10) | 0.0153 | 0.0067 |
| MD_peptide | 19.31 | (5.57) | 17.82 | (6.05) | 16.44 | (5.06) | 18.14 | (5.76) | 3e−04 | <.0001 |
| Abeta1-42/40 | 0.13 | (0.01) | 0.12 | (0.05) | 0.08 | (0.03) | 0.11 | (0.04) | <1e−04 | <.0001 |
| Abeta1-37/40 | 0.11 | (0.02) | 0.09 | (0.02) | 0.09 | (0.02) | 0.09 | (0.02) | <1e−04 | <.0001 |
| Abeta1-38/40 | 0.21 | (0.01) | 0.21 | (0.06) | 0.21 | (0.08) | 0.21 | (0.05) | 0.9483 | 0.3247 |
| Abeta1-39/40 | 0.05 | (0.01) | 0.06 | (0.02) | 0.06 | (0.03) | 0.06 | (0.02) | 0.1652 | 0.1762 |
| Abeta1-43/40 | 0.01 | (0.00) | 0.01 | (0.01) | 0.01 | (0.01) | 0.01 | (0.01) | 3e−04 | <.0001 |
| Abeta1-37/42 | 0.85 | (0.18) | 0.84 | (0.35) | 1.26 | (0.44) | 0.93 | (0.36) | <1e−04 | <.0001 |
| Abeta1-38/42 | 1.69 | (0.30) | 1.98 | (0.75) | 3.03 | (1.23) | 2.08 | (0.90) | <1e−04 | <.0001 |
| Abeta1-39/42 | 0.43 | (0.09) | 0.52 | (0.22) | 0.81 | (0.36) | 0.54 | (0.26) | <1e−04 | <.0001 |
| Abeta1-43/42 | 0.06 | (0.01) | 0.11 | (0.13) | 0.16 | (0.16) | 0.10 | (0.11) | <1e−04 | <.0001 |
| Abeta1-37/43 | 14.06 | (3.28) | 13.32 | (8.48) | 16.80 | (14.03) | 14.31 | (8.61) | 0.0055 | <.0001 |

TABLE 2
Correlation of Abeta isoforms with Thal phase for amyloid plaques (A score)

| | Phase 5 (A3) | NA | Overall | |
|---|---|---|---|---|
| | (N = 32) | (N = 348) | (N = [symbol] ) | P-value |
| Abeta40 | | | | |
| Mean (SD) | 9.38 (2.44) | 11.5 (3.69) | 11.4 (3.68) | 0.0014 |
| Median [Min, Max] | 9.04 [4.94, 16.6] | 11.1 [2.82, 27. [symbol] ] | 10.9 [2.82, 27.3] | |
| Missing | 0 (0%) | 7 (0.8%) | 7 (0.8%) | |
| Abeta42 | | | | |
| Mean (SD) | 0.743 (0.324) | 1.27 (0.614) | 1.28 (0.614) | <1e−04 |
| Median [Min, Max] | 0.697 [0.458, 2.06] | 1.19 [0.223, 4.77] | 1.16 [0.223, 4.77] | |
| Missing | 0 (0%) | 0 (0.7%) | 0 (0.7%) | |
| Abeta37 | | | | |
| Mean (SD) | [symbol] (0.334) | 1.19 (0.421) | 1.09 (0.421) | 7e−04 |
| Median [Min, Max] | 0.796 [0.282, 1.96] | 1.55 [0.249, 3.39] | 1.04 [0.260, 3.39] | |
| Missing | 1 (3.1%) | 16 (1.9%) | 17 (1.9%) | |
| Abeta38 | | | | |
| Mean (SD) | 1.82 (0.645) | 2.43 (1.00) | 2.41 (0.999) | 9e−04 |
| Median [Min, Max] | 1.70 [0.849, 3.76] | 2.28 [0.551, 15.3] | 2.23 [0.561, 16.3] | |
| Missing | 2 (6.3%) | 0 (1.3%) | 11 (1.5%) | |
| Abeta39 | | | | |
| Mean (SD) | 0.455 (0.164) | 0.642 (0.318) | 0.635 (0.314) | 0.001 |
| Median [Min, Max] | 0.410 [0.186, 0.995] | 0.551 [0.142, 4.67] | 0.681 [0.142, 4.67] | |
| Missing | 0 (0%) | 13 (1.5%) | 13 (1.5%) | |
| Abeta43 | | | | |
| Mean (SD) | 0.0896 (0.0584) | 0.106 (0.0989) | 0.105 (0.0977) | 0.6393 |
| Median [Min, Max] | 0.0735 [0.0222, 0.194] | 0.0822 [0.0168, 1.66] | 0.0819 [0.0166, 1.55] | |
| Missing | 0 (0%) | 0 (0.7%) | 6 (0.7%) | |

[symbol]

indicates data missing or illegible when filed

TABLE 3A
Correlation of Abeta isoforms with Braak stage for neurofibrillary
degeneration (B score)

| | Stage V (B3) | Stage VI (B3) | NA | Overall | |
|---|---|---|---|---|---|
| | (N = 5) | (N = 27) | (N = 848) | (N = 880) | P-value |
| Abeta40 | | | | | |
| Mean (SD) | 8.60 (1.26) | 9.52 (2.69) | 11.6 (3.69) | 11.4 (3.68) | 0.0055 |
| Median [Min, Max] | 7.99 [7.74, 19.8] | 9.10 [4.94, 16.6] | 11.1 [2.82, 27.3] | 10.9 [2.82, 27.3] | |
| Missing | 0 (0%) | 0 (0%) | 7 (0.8%) | 7 (0.8%) | |
| Abeta42 | | | | | |
| Mean (SD) | 0.601 (0.048) | 0.787 (0.334) | 1.27 (0.614) | 1.25 (0.614) | <1e−04 |
| Median [Min, Max] | 0.481 [0.460, 0.575] | 0.068 [0.469, 2.06] | 1.19 [0.223, 4.77] | 1.16 [0.223, 4.77] | |
| Missing | 0 (0%) | 0 (0%) | 0 (0.7%) | 0 (0.7%) | |
| Abeta37 | | | | | |
| Mean (SD) | 0.841 (0.115) | 0.637 (0.363) | 1.16 (0.423) | 1.09 (0.421) | 0.0031 |
| Median [Min, Max] | 0.836 [0.723, 0.997] | 0.777 [0.282, 0.997] | 1.05 [0.260, 3.39] | 1.04 [0.260, 3.39] | |
| Missing | 0 (0%) | 1 (3.7%) | 16 (1.9%) | 17 (1.9%) | |
| Abeta38 | | | | | |
| Mean (SD) | 1.83 (0.276) | 1.81 (0.700) | 2.43 (1.00) | 2.41 (0.999) | 0.0041 |
| Median [Min, Max] | 1.67 [1.57, 2.15] | 1.72 [0.049, 3.76] | 2.28 [0.561, 16.3] | 2.23 [0.561, 16.3] | |
| Missing | 0 (0%) | 2 (7.4%) | 11 (1.3%) | 13 (1.5%) | |
| Abeta39 | | | | | |
| Mean (SD) | 0.432 (0.0821) | 0.460 (0.178) | 0.642 (0.318) | 0.635 (0. [symbol] ) | 0.0043 |
| Median [Min, Max] | 0.384 [0.372, 0.660] | 0.419 [0.186, 0.995] | 0.591 [0.142, 4.87] | 0.581 [0.142, 4.87] | |
| Missing | 0 (0%) | 0 (0%) | 13 (1.5%) | 13 (1.5%) | |
| Abeta43 | | | | | |
| Mean (SD) | 0.141 (0.0173) | 0.0000 (0.0534) | 0.106 (0.0989) | 0.105 (0.0977) | 0.2851 |
| Median [Min, Max] | 0.134 [0.123, 0.167] | 0.0516 [0.0222, 0.194] | 0.0822 [0.0166, 1.99] | 0.0819 [0.0166, 1.55] | |
| Missing | 0 (0%) | 0 (0%) | 6 (0.7%) | 6 (0.7%) | |

[symbol]

indicates data missing or illegible when filed

TABLE 3B
Correlation of Abeta isoforms with Braak stage for neurofibrillary
degeneration with non-missing data

| | Stage V (B3) | Stage VI (B3) | Overall | |
|---|---|---|---|---|
| | (N = 5) | (N = 27) | (N = 32) | P-value |
| Abeta40 | | | | |
| Mean (SD) | [symbol] (1.26) | 9.52 (2.69) | 9.38 (2.44) | 0.4462 |
| Median [Min, Max] | 7.99 [7.74, 10.8] | 9.10 [4.94, 16.6] | 9.04 [4.94, 16.6] | |
| Abeta42 | | | | |
| Mean (SD) | 0.601 (0.0483) | 0.787 (0.334) | 0.743 (0.324) | 0.069 |
| Median [Min, Max] | 0.481 [0.460, 0.575] | 0.668 [0.469, 2.06] | 0.657 [0.450, 2.06] | |
| Abeta37 | | | | |
| Mean (SD) | 0.841 (0.115) | 0.637 (0.363) | 0.838 (0.334) | 0.9808 |
| Median [Min, Max] | 0.836 [0.723, 0.997] | 0.777 [0.282, 1.96] | 0.796 [0.282, 1.96] | |
| Missing | 0 (0%) | 1 (3.7%) | 1 (3.1%) | |
| Abeta38 | | | | |
| Mean (SD) | 1.83 (0.276) | 1.81 (0.700) | 1.82 (0.645) | 0.9538 |
| Median [Min, Max] | 1.67 [1.57, 2.15] | 1.72 [0.849, 3.76] | 1.70 [0.849, 3.76] | |
| Missing | 0 (0%) | 2 (7.4%) | 2 (6.3%) | |
| Abeta39 | | | | |
| Mean (SD) | 0.432 (0.0821) | 0.460 (0.176) | 0.455 (0.164) | 0.7317 |
| Median [Min, Max] | 0.384 [0.372, 0.660] | 0.419 [0.186, 0.995] | 0.418 [0.185, 0.895] | |
| Abeta43 | | | | |
| Mean (SD) | 0.141 (0.0173) | 0.0800 (0.0584) | 0.896 (0.0584) | 0.028 |
| Median [Min, Max] | 0.134 [0.123, 0.167] | 0.0616 [0.0222, 0.194] | 0.0735 [0.0222, 0.194] | |

[symbol]

indicates data missing or illegible when filed

TABLE 4
Correlation of Abeta isoforms with Alpha synuclein antibody

| | Phospo-specific | NA | Overall | |
|---|---|---|---|---|
| | (N = 32) | (N = 848) | (N = 880) | P-value |
| Abeta40 | | | | |
| Mean (SD) | 9.38 (2.44) | 11.6 (3.69) | 11.4 (3.68) | 0.0014 |
| Median [Min, Max] | 3.049 [4.94, 16.6] | 11.1 [ [symbol] , 27.3] | 10.9 [2.62, 27.3] | |
| Missing | 0 (0%) | 7 (0.8%) | 7 (0.8%) | |
| Abeta42 | | | | |
| Mean (SD) | 0.743 (0.324) | 1.27 (0.614) | 1.26 (0.014) | <1e−04 |
| Median [Min, Max] | 0.897 [0.460, 2.06] | 1.19 [0.223, 4.77] | 1.16 [0.223, 4.77] | |
| Missing | 0 (0%) | 6 (0.7%) | 1 (3.1%) | |
| Abeta37 | | | | |
| Mean (SD) | 0.038 (0.334) | 1.10 (0.421) | 1.09 (0.421) | 7e−04 |
| Median [Min, Max] | [symbol] [0.282, 1.96] | 1.65 [0.260, 3.39] | 1.04 [0.260, 3.39] | |
| Missing | 1 (3.1%) | 16 (1.9%) | 17 (1.9%) | |
| Abeta38 | | | | |
| Mean (SD) | 1.82 (0.645) | 2.43 (1.00) | 2.41 (0.993) | 9e−04 |
| Median [Min, Max] | 1.70 [0.849, 3.76] | 2.28 [0.551, 16.3] | 2.23 [ [symbol] , [symbol] ] | |
| Missing | 2 (6.3%) | 11 (1.3%) | 2 (6.3%) | |
| Abeta39 | | | | |
| Mean (SD) | [symbol] (0.0.164) | 0.842 (0.318) | 0.636 (0.315) | 0.001 |
| Median [Min, Max] | 0.418 [0.186, 0.995] | 0.591 [0.142, 4.67] | 0.581 [0.142, 0.467] | |
| Missing | 0 (0%) | 13 (1.5%) | 13 (1.5%) | |
| Abeta43 | | | | |
| Mean (SD) | [symbol] (0.0584) | 0.165 (0. [symbol] ) | 0.108 (0.0377) | 0.3593 |
| Median [Min, Max] | 0.0735 [0.0222, 0.194] | 0.0822 [0.5186, 0.166] | 0.0819 [0.0188, 0.166] | |
| Missing | 0 (0%) | 6 (0.7%) | 6 (0.7%) | |

[symbol]

indicates data missing or illegible when filed

TABLE 5A
Correlation of Abeta isoforms with cerebral amyloid angiopathy

| | Mild | Moderate | Severe | NA | Overall | |
|---|---|---|---|---|---|---|
| | [symbol] | (N = 10) | (N = 13) | [symbol] | [symbol] | P-value |
| Abeta40 | | | | | | |
| Mean (SD) | [symbol] | [symbol] | [symbol] | [symbol] | [symbol] | 0.0083 |
| Median [Min, Max] | [symbol] | [symbol] | [symbol] | [symbol] | [symbol] | |
| Missing | [symbol] | [symbol] | [symbol] | [symbol] | [symbol] | |
| Abeta42 | | | | | | |
| Mean (SD) | [symbol] | [symbol] | [symbol] | [symbol] | [symbol] | <1e−04 |
| Median [Min, Max] | [symbol] | [symbol] | [symbol] | [symbol] | [symbol] | |
| Missing | [symbol] | [symbol] | [symbol] | [symbol] | [symbol] | |
| Abeta37 | | | | | | |
| Mean (SD) | [symbol] | [symbol] | [symbol] | [symbol] | [symbol] | 0.0062 |
| Median [Min, Max] | [symbol] | [symbol] | [symbol] | [symbol] | [symbol] | |
| Missing | [symbol] | [symbol] | [symbol] | [symbol] | [symbol] | |
| Abeta38 | | | | | | |
| Mean (SD) | [symbol] | [symbol] | [symbol] | [symbol] | [symbol] | 0.0054 |
| Median [Min, Max] | [symbol] | [symbol] | [symbol] | [symbol] | [symbol] | |
| Missing | [symbol] | [symbol] | [symbol] | [symbol] | [symbol] | |
| Abeta39 | | | | | | |
| Mean (SD) | [symbol] | [symbol] | [symbol] | [symbol] | [symbol] | 0.0122 |
| Median [Min, Max] | [symbol] | [symbol] | [symbol] | [symbol] | [symbol] | |
| Missing | [symbol] | [symbol] | [symbol] | [symbol] | [symbol] | |
| Abeta43 | | | | | | |
| Mean (SD) | [symbol] | [symbol] | [symbol] | [symbol] | [symbol] | 0.2799 |
| Median [Min, Max] | [symbol] | [symbol] | [symbol] | [symbol] | [symbol] | |
| Missing | [symbol] | [symbol] | [symbol] | [symbol] | [symbol] | |

[symbol]

indicates data missing or illegible when filed

TABLE 5B
Correlation of Abeta isoforms with cerebral amyloid angiopathy with non-
missing data

| | Mild | Moderate | Severe | Overall | |
|---|---|---|---|---|---|
| | (N = 9) | (N = 10) | (N = 13) | (N = 32) | P-value |
| Abeta40 | | | | | |
| Mean (SD) | 9.28 (2.72) | 10.5 (3.16) | 6.57 (1.07) | 9.38 (2.44) | 0.1673 |
| Median [Min, Max] | 9.75 [4.94, 14.5] | 10.3 [5.70, 16.6] | 8.54 [7.22, 10.8] | 9.04 [4.94, 16.6] | |
| Abeta42 | | | | | |
| Mean (SD) | 0.883 (0.495) | 0.623 (0.128) | 0.664 (0.177) | 0.743 (0.324) | 0.02 |
| Median [Min, Max] | 0.735 [0.600, 0.206] | 0.620 [0.469, 0.861] | 0.634 [0.450, 0.925] | 0.657 [0.450, 2.06] | |
| Abeta37 | | | | | |
| Mean (SD) | 0.763 (0.182) | 0.936 (0.631) | [symbol] (0.182) | 0.838 (0.334) | 0.6221 |
| Median [Min, Max] | 0.814 [0.472, 0.951] | 0.761 [0.282, 1.96] | 0.836 [0.413, 1.67] | 0.796 [0.282, 1.96] | |
| Missing | 1 (11.1%) | 0 (0%) | 0 (0%) | 1 (3.1%) | |
| Abeta38 | | | | | |
| Mean (SD) | 1.69 (0.436) | 2.15 (0.955) | 1.63 (0.278) | 1.82 (0.645) | 0.1299 |
| Median [Min, Max] | 1.81 [0.943, 2.17] | 2.10 [0.849, 3.76] | 1.61 [1.29, 2.18] | 1.70 [0.849, 3.76] | |
| Missing | 1 (11.1%) | 0 (0%) | 1 (7.7%) | 2 (6.3%) | |
| Abeta39 | | | | | |
| Mean (SD) | 0.476 (0.146) | 0.461 (0.256) | 0.437 (0.0734) | 0.455 (0.164) | 0.8678 |
| Median [Min, Max] | 0.419 [0.315, 0.790] | 0.389 [0.186, 0.995] | 0.440 [0.312, 0.560] | 0.418 [0.186, 0.995] | |
| Abeta43 | | | | | |
| Mean (SD) | 0.451 (0.6285) | 0.123 (0.0667) | 0.8936 (0.0504) | 0.0896 (0.0584) | 0.0103 |
| Median [Min, Max] | 0.0339 [0.0222, 0.101] | 0.162 [0.0245, 0.194] | 0.111 [0.0296, 0.167] | 0.0735 [0.0222, 0.194] | |

[symbol]

indicates data missing or illegible when filed

What is claimed is:

1. A method for measuring one or more Aβ proteoform(s) in a biological sample, the method comprising
(a) providing a biological sample selected from a blood sample or a CSF sample, wherein the biological sample (i) optionally comprises an isotope labeled internal standard of Aβ, and purifying one or more Aβ proteoform(s) to generate an isolated Aβ sample;
(b) removing the supernatant from the isolated Aβ sample by drying to obtain a dry isolated Aβ sample;
(c) resuspending the dry isolated AD sample in a suitable buffer for analyzing the resuspended sample; and
(d) performing liquid chromatography—mass spectrometry with the sample from step (c) comprising one or more Aβ proteoform(s) to detect and measure the amount of the one or more Aβ proteoform(s), wherein the method does not cleave the one or more Aβ proteoform(s).

2. The method of claim 1, wherein the biological sample is CSF.

3. The method of claim 1 or claim 2, wherein the sample is purified by affinity purification.

4. The method of any one of claims 1-3, wherein affinity purification is performed with one or more immobilized ligand(s) that specifically bind Aβ attached to a solid support bead.

5. The method of claim 4, wherein the affinity purification is performed with at least two immobilized ligands, wherein a first ligand specifically binds an epitope within the mid domain of Aβ, and a second ligand binds an epitope within the N-terminus of Aβ.

6. The method of any one of claims 1-5, wherein not cleaving the one or more Aβ proteoform(s) includes not contacting the Aβ proteoform(s) with a protease.

7. The method of any one of claims 1-6, wherein the one or more Aβ proteoform(s) is selected from the group consisting of Aβ1-40, Aβ31-38, Aβ31-37, Aβ1-34, Aβ1-39, Aβ3-39, Aβ31-33, Aβ11-40, Aβ33-40, Aβ31-42, Aβ 1-19, Aβ31-25, Aβ31-30, Aβ31-28, Aβ32-38, Aβ3-38, Aβ3-34, Aβ11-30, Aβ11-33, Aβ11-37, Aβ2-40, Aβ5-40, Aβ11-38, Aβ11-42, Aβ11-34, Aβ7-33, and Aβ1-36.

8. A method to diagnose a subject as having a high risk of conversion to MCI due to AD, the method comprising
(a) providing an isolated AD sample obtained from a subject and measuring, in the isolated Aβ sample, one or more Aβ proteoform(s) chosen from Aβ1-43, and Aβ1-25, and optionally Aβ1-40 and/or Aβ1-42; and
(b) diagnosing the subject as having a high risk of conversion to MCI due to AD when the measured amount significantly deviates from the mean in a control population without brain amyloid plaques as measured by PET imaging and/or Aβx-42/x-40 measurement in CSF.

9. A method to diagnose a subject as having a high risk of conversion to MCI due to AD, the method comprising
(a) providing an isolated Aβ sample obtained from a subject and measuring, in the isolated Aβ sample, one or more Aβ proteoforms chosen from Aβ1-43, and Aβ1-25, and optionally Aβ1-40 and/or Aβ1-42; and
(b) diagnosing the subject as having a high risk of conversion to MCI due to AD when the measured amount significantly deviates from the mean in a control population with a CDR score of 0 and with brain amyloid plaques as measured by PET imaging and/or Aβx-42/x-40 measurement in CSF.

10. A method to diagnose a subject as having a high risk of conversion to MCI due to AD, the method comprising
(a) providing a first and a second isolated Aβ sample obtained from a subject and measuring, in each isolated Aβ sample, one or more Aβ proteoform(s) chosen from Aβ1-43, Aβ1-25, and optionally Aβ11-40 and/or Aβ1-42;
(b) calculating the change in the amount of each Aβ proteoform measured; and
(c) diagnosing the subject as having a high risk of conversion to MCI due to AD when the calculated change(s) significantly deviate from the mean in a control population without brain amyloid plaques as measured by PET imaging and/or Aβx-42/x-40 measurement in CSF or from the mean in a control population with a CDR score of 0 and with brain amyloid plaques as measured by PET imaging and/or Aβx-42/x-40 measurement in CSF.

11. The method of any one of claims 8-10, wherein a decrease in Aβ1-43 levels that significantly deviate from the mean indicate disease progression to MCI due to AD and/or an increase in Aβ1-25 levels that significantly deviate from the mean indicate disease progression to MCI due to AD.

12. The method of any one of claims 8-10, further comprising calculating a ratio between the amount of a first measured AD proteoform and the amount of a second measured Aβ proteoform.

13. The method of claim 12, wherein a ratio of Aβ1-43 and Aβ1-40 is calculated and a decrease in the value of Aβ1-43/Aβ1-40 that significantly deviates from the mean indicates disease progression to MCI due to AD.

14. A method to detect Aβ amyloidosis in a subject, the method comprising
(a) providing an isolated Aβ sample obtained from a subject and measuring, in the isolated Aβ sample, one or more Aβ proteoforms chosen from Aβ11-43, Aβ1-25, Aβ7-33, Aβ11-38, Aβ11-42, Aβ31-37, Aβ32-40, Aβ33-40, Aβ11-30, Aβ31-28, and optionally Aβ1-40 and/or Aβ1-42; and
(b) detecting amyloidosis when the measured amount of Aβ proteoform(s) significantly deviate from the mean in a control population without brain amyloid plaques as measured by PET imaging and/or Aβx-42/x-40 measurement in CSF.

15. The method of claim 14, wherein a decrease in Aβ31-43 levels that significantly deviate from the mean indicate an amyloid positive subject and/or an increase in Aβ1-25 levels indicate an amyloid positive subject and/or an increase in Aβ7-33 levels indicate an amyloid positive subject.

16. The method of claim 14, further comprising calculating a ratio between the amount of a first measured AD proteoform and the amount second measured Aβ proteoform.

17. The method of claim 16, wherein a ratio of Aβ1-43 and Aβ1-40; a ratio of Aβ1-42 and Aβ1-40; a ratio of Aβ1-43 and Aβ11-38; a ratio of Aβ1-43 and Aβ11-42, a ratio of Aβ1-37 and Aβ1-43; a ratio of Aβ2-40 and Aβ1-43; and/or a ratio of Aβ1-42 and Aβ1-28 are calculated.

18. The method of claim 17, wherein a decrease in the value of Aβ1-43/Aβ1-40 and/or Aβ1-42/Aβ1-40 and/or Aβx-42/Aβx-40 and/or Aβ1-43/Aβ11-38 and/or Aβ1-42/Aβ1-28 that significantly deviate from the mean indicate an amyloid positive subject.

19. The method of claim 117 or claim 18, wherein an increase in the value of Aβ1-37/Aβ1-43 and/or Aβ2-40/Aβ1-43 that significantly deviate from the mean indicate an amyloid positive subject.

20. A method for treating a subject in need thereof, the method comprising
(a) providing an isolated Aβ sample obtained from a subject and measuring, in the isolated Aβ sample, one or more Aβ proteoforms chosen from Aβ1-43, Aβ1-25, Aβ7-33, Aβ11-38, Aβ11-42, Aβ1-37, Aβ2-40, Aβ3-40, Aβ11-30, Aβ31-28, and optionally Aβ1-40 and/or Aβ1-42; and
(b) administering a pharmaceutical composition to the subject when the measured amount of Aβ proteoform(s) significantly deviate from the mean in a control population without brain amyloid plaques as measured by PET imaging and/or Aβx-42/x-40 measurement in CSF.

21. The method of claim 20, further comprising calculating a ratio between the amount of a first measured Aβ proteoform and the amount second measured Aβ proteoform.

22. The method of claim 21, wherein a ratio of Aβ1-43 and Aβ31-40; a ratio of Aβ1-42 and Aβ1-40; a ratio of Aβ1-43 and Aβ11-38; a ratio of Aβ1-43 and Aβ11-42, a ratio of Aβ1-37 and Aβ31-43; a ratio of Aβ2-40 and Aβ1-43; and/or a ratio of Aβ1-42 and Aβ31-28 are calculated.

23. The method of claim 21, administering a pharmaceutical composition to the subject when a decrease in the value of Aβ11-43/Aβ1-40 and/or Aβ1-42/Aβ31-40 and/or Aβx-42/Aβx-40 and/or Aβ1-43/Aβ11-38 and/or Aβ1-42/Aβ1-28 that significantly deviate from the mean in a control population.

24. The method of claim 22, administering a pharmaceutical composition to the subject when an increase in the value of Aβ1-37/Aβ1-43 and/or Aβ2-40/Aβ1-43 that significantly deviate from the mean indicate an amyloid positive subject.
